(19) United States
(12) Patent Application Publication
(10) Pub. No.: US 2021/0077477 A1
(43) Pub. Date: Mar. 18, 2021

(54) MICROGLIA MODULATORS FOR USE IN TREATMENT OF DEPRESSION

(71) Applicant: Raz YIRMIYA, Jerusalem (IL)

(72) Inventor: Raz YIRMIYA, Jerusalem (IL)

(21) Appl. No.: 16/963,732

(22) PCT Filed: Mar. 27, 2019
(86) PCT No.: PCT/IL2019/050356

§ 371 Date: Jul. 21, 2020

Related U.S. Application Data
(60) Provisional application No. 62/648,465, filed on Mar. 27, 2018.

(51) Int. Cl. A61K 31/444; A61P 25/24; A61K 31/65; C07K 16/28; A61K 39/395

(52) U.S. Cl. CPC A61K 31/444; A61P 25/24; A61K 2039/545; C07K 16/2803; A61K 39/3955; A61K 31/65

(57) ABSTRACT

The present invention relates to methods for treating a depression condition in a subject, including administering to the subject at least one microglial modulator or a combination thereof. Further provided are methods using a microglial modulator(s) in combination with non-invasive brain stimulation (NIBS), such as electroconvulsive therapy (ECT).

### CROSS-REFERENCE TO RELATED APPLICATIONS

This application claims the benefit of priority of U.S. Provisional Patent Application No. 62/648,465, filed Mar. 27, 2018, the contents of which is incorporated herein by reference in its entirety.

### FIELD OF INVENTION

The present invention is in the field of neuropharmacology, and in some embodiments thereof, is directed to antidepressant drugs and procedures.

### BACKGROUND OF THE INVENTION

Despite impressive progress in understanding the molecular, cellular and circuit-level correlates of depression, the biological mechanisms that causally underlie this disease are still unclear, hindering the development of effective preventive and therapeutic procedures. On this note, effectivity of SSRIs, the most popular class of antidepressant drugs, is limited, with a portion of the population showing lack of treatment efficacy and/or SSRIs-resistance. Accordingly, a method for treating a subject resistant to SSRI therapy is greatly needed.

One possible reason for the slow progress in developing novel and effective antidepressants is that almost all research in this area focuses on the involvement of abnormalities in neuronal functioning, whereas the involvement of other systems, including the immune system, in general, and brain microglia—the representatives of the immune system in the brain, in particular, was not thoroughly examined.

The association between depression and immune alterations has been known for decades. Over the past two decades it has become evident that in some depressed patients the innate immune system is activated, as reflected for example by elevated levels of pro-inflammatory cytokines. With this respect, some studies supported the concept of using anti-inflammatory agents, showing beneficial effects of NSAIDs or TNF-α blockade in depressed patients. However, in some studies the use of such drugs proved to be detrimental for depression. For example, TNF-α blockade was found to exacerbate depression in some patients, and NSAIDs were shown to decrease the anti-depressive activity of SSRIs such as fluoxetine. Furthermore, in some depressed patients and models of depression in rodents the innate immune system and particularly microglia cells are suppressed and degenerated.

Electroconvulsive therapy (ECT), in which electric currents are passed through the brain, was found to modify brain's chemistry and promote neurogenesis, thus may rapidly ameliorate symptoms of mental illnesses, including depression and schizophrenia. Although quite effective, ECT suffers a stigma based mainly on its early historical treatments in which overdosing currents were applied to an un-anaesthetized subject, resulting with bone fractures, memory loss, or other serious side effects.

### SUMMARY OF THE INVENTION

The present invention relates to methods and compositions for treating depression in a subject in need thereof. In some embodiments, there is provided use or administration of at least one microglia modulator or a combination thereof. The present invention is based, in part, on the finding that a microglia modulator as described herein was found to be more effective than a SSRI drug, e.g., escitalopram. Surprisingly, this therapeutic effect was found to be reversed when the microglia modulator was applied concomitantly with escitalopram. Accordingly the present invention provides a microglia modulator as a replacement for SSRIs therapy either as a first line therapy or specifically in S SRI-resistant or non-responding subjects (e.g., as a second line therapy).

According to one aspect, there is provided a method for treating or attenuating a depressive disorder in a selective serotonin reuptake inhibitor (SSRI) non-treated subject, the method comprising administering to the subject a pharmaceutical composition comprising a therapeutically effective amount of at least one compound inhibiting a molecule selected from the group consisting of: lymphocyte-activation gene 3 (LAG-3), cluster of differentiation molecule 180 (CD180), tryptophan 2,3-dioxygenase (TDO2), cluster of differentiation molecule 86 (CD86/B7-2), programmed cell death ligand 1 (PD-L1), and Phospholipase A2 Group IVE (PLA2G4E); and at least one pharmaceutically acceptable carrier or diluent; thereby treating or attenuating the depressive disorder in the subject.

According to another aspect, there is provided a method for increasing the therapeutic response to non-invasive brain stimulation (NIBS) therapy in a subject in need thereof, the method comprising administering to the subject a pharmaceutical composition comprising a therapeutically effective amount of at least one microglia modulator and at least one pharmaceutically acceptable carrier or diluent.

According to another aspect, there is provided a method for treating or attenuating schizophrenia or symptoms thereof in a subject in need thereof, the method comprising: administering to the subject a pharmaceutical composition comprising therapeutically effective amount of at least one microglia modulator and at least one pharmaceutically acceptable carrier or diluent; thereby treating schizophrenia in the subject.

In some embodiments, the method further comprises the step of administering a second microglial activator to said subject.

In some embodiments, the second microglial activator is selected from the group consisting of: Macrophage colony-stimulating factor (M-CSF), Granulocyte macrophage colony-stimulating factor (GM-CSF), Interleukin 34 (IL-34), Granulocyte colony-stimulating factor (G-CSF), soluble LAG-3, and CX3C chemokine receptor 1 (CX3CR1) blockers.

In some embodiments, the method further comprises selecting a subject having an increased level of at least one transcript or a protein product thereof compared to a baseline, wherein the transcript or a protein product thereof is selected from the group consisting of: LAG-3, CD180, TDO2, CD86/B7-2, PD-L1, and PLA2G4E.

In some embodiments, the transcript or a protein product thereof is detected in a sample of the subject, wherein the sample comprises: whole blood, peripheral blood mononuclear cells (PBMCs), isolated T cells, isolated dendritic cells, or isolated monocytes.

In some embodiments, the method further comprises selecting a subject having a low inflammatory state.

In some embodiments, low inflammatory state is reflected by plasma C-reactive protein (CRP) lower than 3 mg/L.

In some embodiments, selecting a subject having a low inflammatory state is determining the plasma level of at least one inflammatory marker selected from CRP, IL-6 and TNFα, wherein a level of any one of: (i) less than 3 mg/L CRP, (ii) less than 2.0 pg/ml IL-6, (iii) less than 3.8 pg/ml TNFα, and (iv) combination thereof, indicates the subject has a low neuroinflammatory state suitable for treatment by the inhibitory-compound.

In some embodiments, the depressive disorder is selected from the group consisting of: unipolar major depressive episode, major depressive disorder, dysthymic disorder, treatment-resistant depression, bipolar depression, adjustment disorder with depressed mood, cyclothymic disorder, melancholic depression, psychotic depression, post-schizophrenic depression, depression due to a general medical condition, post-viral fatigue syndrome, and chronic fatigue syndrome.

In some embodiments, the at least one compound targets CD180.

In some embodiments, the at least one compound targets PLA2G4E.

In some embodiments, the compound is selected from the group consisting of: a polynucleotide, a peptide, a peptidomimetic, a carbohydrate, a lipid, a small organic molecule, and an inorganic molecule.

In some embodiments, the method further comprises a step of applying a non-invasive brain stimulation (NIBS) to the subject.

In some embodiments, the increased therapeutic response to NIBS is measured by a reduction in one or more effects selected from the group consisting of: acute confusional state, tachycardia, atrial arrhythmia, ventricular arrhythmia, hypertension, asystole, muscle pain, fatigue, headaches, nausea, and amnesia.

In some embodiments, the increased therapeutic response to NIBS is measured by a reduction in the number, length or frequency of NIBS treatments necessary to achieve a desired therapeutic effect, or any combination thereof.

In some embodiments, the increased therapeutic response to NIBS is measured by a reduction of stimulus intensity, stimulus dosage necessary to achieve a desired therapeutic effect, or any combination thereof.

In some embodiments, the composition is administered 1 to 72 hours prior to applying NIBS.

In some embodiments, the ratio of microglia modulator administration to NIBS application ranges from 10:1 to 1:10.

In some embodiments, the NIBS is selected from the group consisting of: electroconvulsive therapy (ECT), repetitive transcranial magnetic stimulation (rTMS), deep TMS, cranial electrotherapy stimulation (CES), transcranial direct current stimulation (tDCS), transcranial random noise stimulation (tRNS), and reduced impedance non-invasive cortical electrostimulation (RINCE).

In some embodiments, the subject is afflicted with a disorder selected from the group consisting of: unipolar major depressive episode, major depressive disorder, dysthymic disorder, treatment-resistant depression, bipolar depression, adjustment disorder with depressed mood, cyclothymic disorder, melancholic depression, psychotic depression, schizophrenia, post-schizophrenic depression, depression due to a general medical condition, post-viral fatigue syndrome, and chronic fatigue syndrome.

In some embodiments, the symptoms are selected from the group consisting of: depression, anhedonia, apathy, catatonia and social problems, and withdrawal.

In some embodiments, the subject has a low number of microglia cells, low activity of microglia cells, or both.

In some embodiments, the microglia modulator is an inhibitory-compound targeting a molecule selected from the group consisting of: LAG-3, CD180, TDO2, B7-2, PD-L1, and PLA2G4E.

In some embodiments, the microglia modulator is administered to the subject at a dosage of 0.01 to 100 mg/kg body weight.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIGS. 1A-1G are vertical bar graphs and micrographs describing the effects of chronic unpredictable stress (CUS) exposure and ECT (electroconvulsive therapy; or SHAM treatment) on microglial morphology. (1A) Examination of the effects of CUS exposure and ECT on density (number/mm²) of microglia in the hippocampal dentate gyrus (DG). CUS exposure induced a significant reduction in the number of hippocampal microglia, compared to control non-stressed mice, whereas treatment of CUS-exposed mice with ECT (3 times/week for 2.5 weeks) reversed this effect. This finding was reflected by a significant overall group difference (F(2,65)=4.44, p<0.02, n=20-26 DG images from five different brains per condition), as well as by post-hoc tests showing significant differences between the SHAM-treated mice and the other two groups (#p<0.05). (1B) CUS-exposed, ECT-treated mice showed significantly enlarged ionized calcium-binding adapter molecule 1 (IBA1)-stained cell bodies of microglia in the DG of the hippocampus (which is a characteristic of microglial activation). This was reflected by a significant overall group difference (F2, 308=3.29, p<0.04, n=94-109 microglia from the DG region, images were taken from 5-7 different brains per condition), as well as by post-hoc tests showing significant differences between the ECT-treated mice and control (p<0.02). (1C) CUS-exposed, SHAM-treated mice exhibited a significant reduction in the area of IBA1-positive cells, which was reversed by ECT. This was reflected by a significant overall group difference (F2, 305=3.2, p<0.05, n=90-110 microglia from the DG region, images were taken from 5-7 different brains per condition), as well as by post-hoc tests showing a significant difference between the SHAM-treated mice and the control group (p<0.03), but no difference between the CUS-exposed ECT-treated group and the control group. (1D) CUS exposure produced an overall decrease in the length of microglial processes in both SHAM and ECT-treated mice. This was reflected by a significant group difference (F2, 211=3.45, p<0.05, n=60-84 microglia DG cells, 5 brains/condition), as well as by a significant difference between the non-stressed control and each of the CUS-exposed groups (p<0.03). (1E-1G) are representative micrographs of microglia in the DG of the hippocampus of a control (CON; 1E) non-stressed mouse, as well as CUS-exposed mice treated with either SHAM (1F) or ECT (1G).

FIGS. 2A-2J are illustrations, graphs and micrographs demonstrating how depletion of brain microglia blocks the anti-depressive and neurogenesis enhancing effects of ECT. (2A) an illustration of a non-limiting time line of the experiment. Following 4 weeks exposure to either a normal control diet (CDiet) or a diet containing PLX5622 (an antagonist of the CSF-1 receptor; essential for microglial survival), i.e., after attainment of microglial depletion in the PLX5562-treated animals, subjects from the two diet groups were exposed to a Chronic Unpredictable Stress (CUS) schedule. Another group of subjects administered with the control diet did not undergo the CUS procedure and served as an untreated (no-CUS) control group. At the beginning of the sixth week of the CUS exposure, following verification of CUS-induced depressive-like symptoms, mice were further divided into two sub-groups, administered with either ECT (3-times per week for 2.5 weeks) or sham treatment. (2B) is a fluorescent image of a hippocampal dentate gyrus (DG) of a mouse fed on normal control diet (CDiet). (2C) is a fluorescent image of the DG area of a mouse fed with the PLX5622 diet (PLX), demonstrating the near-complete depletion of microglia (green IBA-1-labeled cells). (2D) is a bar graph describing the suppression of sucrose preference (anhedonia) in both CDiet and PLX-treated mice exposed to CUS for 5 weeks. Non-stressed mice treated with either CDiet or PLX displayed similar levels of sucrose preference, as well as a similar CUS-induced reduction in sucrose preference. This finding was reflected by a significant main effect of CUS (F1, 60=21.433, p<0.0001) (n=8-20/group). Post-hoc analyses revealed that in the CUS-exposed CDiet and PLX groups sucrose preference was significantly lower than the preference in the respective no-CUS groups 4(32)=3.419, and t(23)=3.722, respectively, p<0.01). (2E) is a bar graph describing the suppression of social exploration in both CDiet and PLX-treated mice exposed to CUS for 5 weeks. Mice treated with either CDiet or PLX displayed similar levels of social exploration, as well as a similar CUS-induced reduction in social exploration. This finding was reflected by a significant main effect of CUS (F1, 64=6.993, p<0.01) (n=8-20/group). (2F) is a bar graph describing the attenuation of the effect of ECT on sucrose preference by microglia depletion. Following CUS exposure, control diet mice that were treated by SHAM ECT (the ECT procedure without passing electroconvulsive shock) displayed the expected decrease in sucrose preference (a model of anhedonia). ECT reversed this effect. In mice fed with the PLX diet, ECT produced only a small increase in sucrose preference. These findings were reflected by a significant group effect (F(1, 22)=4.699, p<0.05; n=6-11/group). Post-hoc analysis confirmed that ECT increased sucrose preference in both the CON (t(11)=−2.84, p<0.05) and PLX (t(11)=−2.822, p<0.05) groups. However, the effect of ECT in the PLX-treated mice was significantly lower than in the CDiet mice (412)=3.25, p<0.05)). (2G) is a bar graph describing the attenuation of the effect of ECT on social exploration by microglia depletion. ECT produced an overall increase in social exploration (F1,22=13.925, p<0.001) (n=6-11/group). This increase was more pronounced in the CDiet-treated mice, but the difference between the effects of ECT in the two diet groups did not reach statistical significance. (2H) is a bar graph describing the blockade of the effect of ECT on despair-like behavior in the forced swim (Porsolt) test by microglia depletion. In non-stressed mice, the basal levels of immobility in the Porsolt forced swim test was similar in the CDiet and PLX groups. In CUS-exposed mice, ECT attenuated the effects of CUS. This finding was reflected by a significant difference among the groups (F(1,87)=3.56, p<0.01) (n=6-11/group). Post hoc analysis revealed that ECT-treated PLX mice displayed significantly greater forced swim immobility than CDiet-treated mice (p<0.001), reflecting the abrogation of the anti-depressive effect of ECT in this test in microglia-depleted mice. (2I) is a bar graph describing the complete blockade of the effect of ECT on hippocampal neurogenesis by microglia depletion. CUS-exposed mice treated with either CDiet or PLX displayed similar levels of neurogenesis (number of DCX-stained cells) in the hippocampal DG. However, whereas in mice on the CDiet ECT significantly increased neurogenesis, in mice on the PLX diet ECT significantly reduced the levels of neurogenesis. These findings were reflected by a significant interaction ((F1,87=20.1, p<0.001) (n=3-6/group), #p<0.05 between ECT and SHAM per each diet condition. (2J) is representative pictures of DCX staining (red) in the DG of SHAM- or ECT-treated depressed-like mice consuming CDiet or PLX diet. Microglia are stained green (IBA1), and nuclei stained blue (DAPI).

FIGS. 3A-3H are vertical bar graphs describing the validation by qPCR of immune/microglial modulating genes showing significant ECT-induced transcriptional regulation changes in the RNA-Seq analysis. As shown, all ECT-induced transcriptional effects depended on the presence of microglia (i.e., did not occur in PLX5622-treated (microglia-depleted) subjects). (3A) Lag-3 gene expression validation revealed a main effect of Diet (F(1,17)=46.8, p<0.001; n=5-6/condition), with low expression levels in the PLX-treated mice. In addition, there was a significant interaction between Diet and Treatment (F(1,17)=4.43 p<0.05; n=5-6/condition). Post-hoc tests revealed a significant reduction in Lag-3 expression in CDiet mice subjected to ECT, compared to SHAM-treated mice (t(9)=2.3, p<0.05), but no effect of ECT in PLX-treated mice. (3B) Cd180 gene expression validation revealed a main effect of Diet (F(1,17)=47.3, P<0.001; n=5-6/condition), with lower expression levels in the PLX-treated mice. Similar to the finding in the RNA-Seq analysis (Table 1), the expression of Cd180 were reduced by ECT in CDiet animals, but this finding did not reach statistical significance. (3C) Tdo2 gene expression validation revealed a significant main effect of Diet (F(1,16)=4.6, p<0.05; n=5/condition). Post-hoc tests revealed a significant decrease in Tdo2 expression in CDiet mice subjected to ECT, compared to SHAM-treated mice (t(8)=2.56, p<0.05), but no effect of ECT in PLX-treated mice. (3D) Pla2g4e gene expression validation revealed a main effect of diet (F(1,17)=10.44, P<0.01) (n=5-6/condition). Post-hoc tests revealed a significant decrease in Pla2g4e expression in CDiet mice subjected to ECT compared to SHAM-treated mice (t(9)=(3.63), p<0.01), but no effect of ECT in PLX-treated mice. (3E) Sox11 gene expression validation revealed a main effect of Treatment (F1,16=5.927 p<0.05), with a significant increase in Sox11 expression in CDiet mice subjected to ECT, compared to SHAM-treated mice (t (9)=(3.14), p<0.05). (3F) Dopamine receptor D1 (Drd1) gene expression validation revealed a main effect of Diet (F1,17=6.14, p<0.05) and a main effect of Treatment (F1,16=7.08, p<0.05). Post hoc test revealed a significant increase in Drd1 expression in CDiet mice subjected to ECT, compared to SHAM-treated (t(9)=(−2.52), p<0.05). (3G) Iba1 and (3H) P2ry12 gene expression validation revealed a main effect of Diet (F1,17=436.0 and F1,17=625.8, respectively, p<0.001) (n=5-6/condition, for this and for all other validations presented in this figure), with low levels of expression in PLX-treated mice.

FIGS. 4A-4G are illustrations, graphs and micrographs demonstrating concurrent administration of ECT together with minocycline (a drug that blocks the ability of microglia to undergo activation) prevents the therapeutic effects of ECT on anhedonia (a core depressive symptoms) and on reduced hippocampal neurogenesis (considered an important biological mechanism of depression and antidepressants). (4A) an illustration of a non-limiting time line of the experiment. Following 5 weeks exposure to CUS or to non-stress control (CON) period, and verification of CUS-induced depressive-like symptoms, half of the mice within each group were initiated on minocycline (MINO; administered in the drinking water)) and the other half on water (VEH) only. After 3 days, half the mice in each group the CUS-exposed mice from each group were further divided into two sub-groups, administered with either ECT (3-times per week for 2.5 weeks) or sham treatment. (4B) a graph showing that following exposure to five weeks of CUS, sucrose preference (an established model of hedonic behavior in mice) was significantly reduced (t(31)=(−8.57), p<0.0001) (n=6-7/condition). (4C) a graph showing that following ECT, CUS-exposed mice on vehicle showed restoration of sucrose preference whereas minocycline (MINO)-treated mice showed no therapeutic effect of ECT. These findings were reflected by a significant group effect (F(4, 28)=5.96, p<0.001, n=6-7 per group). Post-hoc analysis revealed a significant ECT-induced increase in sucrose preference in water-drinking but not MINO-treated mice (p<0.05). (4D) a showing that in the Porsolt forced swim test, ECT produced an overall reduction in immobility time, reflected by a significant main effect of ECT (F1,21=4.33 p<0.05, n=6-7 per group). (4E) a graph showing that CUS exposure produced an ECT-reversible suppression in neurogenesis in water-drinking, but not MINO-treated mice. This was reflected by a significant overall group difference in the number of Doublecortin (DCX)-positive cells in the DG (F(4, 25)=5.29, p<0.005, n=5-7 brains/condition), as well as by post hoc analysis showing that neurogenesis was significantly lower in the water-drinking SHAM-treated group, MINO-drinking SHAM-treated group, and MINO-drinking ECT-treated groups than in either the non-stressed control or the water-drinking ECT-treated groups (p<0.01). (4F) a graph showing that ECT increased the number of contacts between microglia and new-born neurons in water-drinking but not MINO-treated mice. This finding was reflected by a significant overall group difference (F(4, 830)=4.46, p<0.001, n=144-218 DG cells, 5-6 brains/condition), as well as post hoc analysis showing a significantly high number of contacts between microglial processes and DCX-positive cells in the water drinking ECT-treated group as compared to either the non-stressed controls (p<0.001) or all other treatment groups (p<0.05). (4G) is representative micrographs of the hippocampal DG, depicting the reduced number of contacts between microglia (stained with green IBA-1) and newborn (neurogenic) neurons (stained with red DCX).

FIGS. 5A-5E are fluorescent micrographs of the LAG-3 protein expression by microglia within the dentate gyrus of the hippocampus. (5A) Immunohistochemical staining of the hippocampal DG demonstrated that LAG-3 (red) protein is localized almost exclusively to microglia (Iba-1; green). Cell nuclei were also stained (DAPI; blue). Notably, all microglia were found to be LAG-3 labeled. (5B-5D) Immunohistochemical staining of a typical microglia. LAG-3 (red) was shown to be expressed on the microglial cell membrane, both of the soma and the processes. (5E) A human microglia cell double-stained with both LAG-3 (red) and the microglia marker IBA-1 (green).

FIGS. 6A-6D is a vertical bar graph and fluorescent micrographs demonstrating that ECT normalizes the CUS-induced higher levels of microglial LAG-3 protein. (6A) CUS induced a significant increase in LAG-3 (average intensity), reflected by an overall group difference (F(2, 187)=35.7, p<0.001, n=30-80 DG microglia, 5 brains/condition), and by a significant difference between the control group and the SHAM and ECT groups (#p<0.01). Additionally, ECT treatment significantly reduced the levels of LAG-3 intensity compared to the ECT treated group (* p<0.01). (6B-6D) are representative fluorescent micrographs of microglia cells form control (CON; 6B) mice or CUS-exposed mice treated with SHAM (6C) or ECT (6D). In the IBA-labeled (green) microglia, LAG-3 staining intensity (red) is greater in the microglia from a CUS-exposed SHAM-treated mouse than in microglia from a CON mouse and a CUS-exposed ECT-treated mouse.

FIGS. 7A-7C are illustrations and demonstrating that a single treatment with a LAG-3 antibody ameliorates the anhedonia and despair (two core symptoms of depression) induced by CUS exposure. (7A) an illustration of a non-limiting time line of the experiment. Mice were exposed to CUS for 5 weeks or to no stress (CON) and were then tested in the sucrose preference test (before treatment). After verification that CUS induced a reduction in sucrose preference at this time, mice were treated with either anti-LAG-3 antibody or with a control IgG antibody, and CUS exposure continued in the relevant groups. Sucrose preference was tested again 3 days following the injection (after treatment) and the Porsolt forced swim test at 5 days following the injection. (7B) a graph showing that the CON (no stress) groups, as well as the CUS-exposed group treated with IgG showed no change from before to after the treatment. In contrast, the CUS-exposed group treated with the Anti-LAG-3 antibody showed a significant increase in sucrose preference, reflecting the reversal of anhedonia. These findings were reflected by a significant 2-way interaction between (exposure (CUS/CON) by time (before/after treatment) as well as an interaction between treatment (anti-IgG/anti LAG-3) by time (before/after treatment) in a repeated measures ANOVA (F(1,26)=5.3 and 4.23, respectively, p<0.05), as well as by a significant effect of anti-LAG-3 Ab in the CUS-exposed mice in post-hoc test (#p<0.01). (7C) is a graph showing that in the IgG-treated group, CUS-exposed mice showed high levels of inactivity in the FST, compared with non-stressed (CON) mice. In contract, no difference was shown by CUS-exposed mice treated by the anti-LAG-3 antibody (LAG-3). These findings were reflected by a significant two-way interaction (exposure (CUS/CON) by treatment (anti-IgG/anti LAG-3) (F(1,20)=5.05, p<0.05), as well as by a significant effect of anti-LAG-3 Ab in the CUS-exposed mice #p<0.01).

FIGS. 8A-8C are illustrations and graphs demonstrating that chronic treatment with a LAG-3 antibody ameliorates CUS-induced anhedonia and social withdrawal (two core symptoms of depression) with more efficacy than the SSRI drug escitalopram (Cipralex). (8A) Time line of the experiment. Mice were exposed to CUS for 5 weeks and were then tested in the sucrose preference and social exploration (SE) tests (before treatment). After verification that CUS induced a reduction in sucrose preference and SE at this time (as compared with levels before the initiation of CUS), mice were treated with either anti-LAG-3 antibody or with a control IgG antibody, injected (i.p.) every 4 days for a total of 6 injection (i.e., in a regimen similar to ECT, over a 3-weeks period). Each of these groups was subdivided into two groups, injected (daily) with either Cipralex (CIP) or saline vehicle (VEH). (8B) Repeated-measures ANOVA, with the antibody (Anti-LAG-3 vs. IgG) and antidepressant (CIP vs. VEH) as between subjects factors and time (before to after the treatment) as a repeated-measures, within-subject factor, revealed a significant 3-way interaction (F1,37=5.946, p<0.02), reflecting the differential effect of the Anti-LAG antibody in the various groups. Post-hoc tests demonstrated that sucrose preference was significantly elevated only after treatment with the anti-LAG-3 antibody by itself (i.e., in the LAG+VEH group) (P<0.001), but not in any other group. (8C) A similar repeated-measures ANOVA on the findings in the SE test also revealed a significant a significant 3-way interaction (F(1,37)=5.354, p<0.03), reflecting the differential effect of the Anti-LAG-3 antibody and of escitalopram in the various groups. Post-hoc analysis revealed a significant increase in SE following treatment with the LAG-3+VEH as well as following IgG+CIP, demonstrating the efficacy of the LAG-3 antibody and of escitalopram treatments by themselves, which is abrogated when both are administered together.

### DETAILED DESCRIPTION OF THE INVENTION

The present invention relates to methods and compositions for treating a depression in a subject in need thereof. In some embodiments, there is provided at least one microglia modulator for treatment of psychiatric condition.

In some embodiments, the subject is a non-SSRI-treated subject.

In some embodiments, a non-SSRI-treated subject is a subject not being treated with SSRI drug.

In some embodiments, a non-SSRI-treated subject is a subject that cannot be treated with a SSRI drug.

In some embodiments, a non-SSRI-treated subject is a subject having resistance to a SSRI drug.

In some embodiments, the subject has been treated with S SRI, but therapy was discontinued. In some embodiments, SSRI therapy discontinuation is attributed to adverse effects.

In some embodiments, SSRI therapy discontinuation is attributed directly to adverse effects.

In some embodiments, SSRI therapy discontinuation is not directly attributed to the therapy.

In some embodiments, SSRI therapy discontinuation which is not directly attributed to the therapy results from cross-reactivity with other therapy or drugs consumed, prescribed, applied, or any combination thereof, by the subject.

In some embodiments, there is provided a combination therapy comprising use of a microglia modulator and a non-invasive brain stimulation therapy (NIBS), such as for treatment of a psychiatric condition.

In some embodiments, the present invention is directed to methods and compositions for treating Schizophrenia in a subject in need thereof.

In some embodiments, methods of the present invention comprise the use of at least one microglia modulator in a composition with at least one pharmaceutically acceptable carrier or diluent.

In some embodiments, the present invention comprises methods for treating or attenuating a depressive disorder in a subject having a low peripheral inflammatory or neuroinflammatory status.

### Microglia Modulator

Microglial activation refers to the fact that when infection, injury or disease occur in the brain and affect nerve cells, microglia in the central nervous system become “active,” causing inflammation in the brain, similar to the manner in which white blood cells act in the rest of the body. Under some conditions, microglia act like the monocyte phagocytic system. Activated microglia can generate large quantities of inflammatory cytokines, as well as superoxide anions, with hydroxyl radicals, singlet oxygen species and hydrogen peroxide being a downstream product, any of which can be assayed in the preparations utilized in such methods of the invention.

Reactive microglia may be characterized by at least one of the following characteristics: 1) their cell bodies becoming larger, their processes becoming shorter and thicker, 2) an increase in the staining for several molecular activation markers, including Iba-1) proliferation and clustering, 4) production and secretion of inflammatory mediators, including pro-inflammatory (e.g., interleukin (IL)-1, IL-6 and tumor necrosis factor-α) and anti-inflammatory (e.g., IL-10, IL-1ra) cytokines, as well as additional inflammatory mediators (e.g., prostaglandins), 5) production and secretion of various neuroprotective factors, including brain-derived neurotrophic factor (BDNF) and insulin growth factor-1 (IGF-1), 6) production and secretion of chemo-attractive factors (chemokines), which recruit microglia from within the brain to specific brain locations and facilitate the infiltration of peripheral immune cells, for example, white blood cells, as compared to that found in the non-reactive state. In some embodiments microglial activation is determined in at least one brain region or area, such as in the hippocampal dentate gyrus (DG), in the prelimbic cortex or in any depression-related area. In some embodiments, microglia activation is characterized based on mRNA or protein expression of microglia checkpoints, such as LAG-3 (Accession number NP_002277.4) and/or CD180 (Accession number NP_005573.2). In some embodiments, microglia activation is determined in case when expression levels of microglia checkpoints, such as LAG-3 and/or CD180 are lower than normal or baseline.

The term “microglia modulator” refers to a compound that may be a nucleic acid-based molecule, amino acid-based molecule or a small organic molecule that causes modulation (e.g., activation) of microglia as will be defined below. In some embodiment, a microglia modulator is a hydrophobic molecule. In some embodiment, a hydrophobic molecule is a lipid. In some embodiments, a microglia modulator is an inhibitory-compound. The modulator may be an isolated full molecule, a fragment or a variant of the molecule as long as it causes microglia modulation (e.g., activation). A microglia activator may cause the effect of microglia activation including but not limited to by acting directly on the microglia or by causing production, expression, secretion, of another agent effecting microglia activation.

In some embodiments, a microglia activator is an inhibitory-compound that removes, breaks, bypasses, or circumvents a microglia checkpoint. As defined herein, the term “inhibitory” refers to a molecule capable of inhibiting or reducing the activity of a specific target. In some embodiments, inhibiting or reducing the activity of a specific target is by at least 10%, 30%, 50%, 75%, 150%, 500%, or 1,000%, or any value and range therebetween. Each possibility represents a separate embodiment of the invention. In some embodiments, inhibiting or reducing the activity of a specific target is by 1-10%, 5-30%, 20-50%, 35-75%, 70-150%, 100-500%, or 250-1,000%. Each possibility represents a separate embodiment of the invention. In some embodiments, inhibiting or reducing the activity of a specific target is by at least 1.5-fold, 2-fold, 5-fold, 10-fold, 50-fold, 100-fold, 500-fold, or 1,000-fold, or any value and range therebetween. Each possibility represents a separate embodiment of the invention.

In some embodiments, the microglia activator increases at least one of the following, all being indicative of microglia activation: increase in hippocampal microglia number as well as increase in number of proliferating microglia (as determined for example by microglia labeled with BrdU); reversal or decrease in dystrophic changes in microglia and increase of their cell body size and processes size and length; and an increase of the expression of activation markers (including Iba-1, MHC class II, P2Y12, CD1 lb) and the production of inflammatory cytokines (including TNF-alpha, IL-1-beta, IL-6, interferon-gamma, M-CSF, GM-CSF).

Non-limiting examples of microglia modulators that may be used in the method and composition of the invention include blocking compounds of: lymphocyte-activation gene 3 (LAG-3), cluster of differentiation molecule (CD180), tryptophan 2,3-dioxygenase (TDO2; Accession number NP_005642.1), cluster of differentiation molecule 86 (CD86/B7-2; Accession number CAG46642.1) and programmed cell death protein 1 (PD-L1; Accession numbers NP_054862.1, NP_001254635.1, or NP_001300958.1), and inhibitors/antagonists of the activity of Phospholipase A2 Group IV E (PLA24E; Accession number Q3MJ16).

As used herein, “a microglia modulator blocking LAG-3” comprises an anti-LAG-3 antibody. According to the present invention, any antibody having specific binding affinity to human LAG-3, is applicable. In some embodiments, having specific binding affinity comprises blocking LAG-3 activity, inhibiting LAG-3 activity, reducing LAG-3 activity, or any equivalent thereof. Anti-LAG-3 antibodies are commercially available, such as LEAF™ Purified anti-mouse CD223 (Biolegend), the use of which has been exemplified hereinbelow.

As defined herein, the term “targeting” refers to having increased binding affinity. In some embodiments, increased binding affinity as used herein is by at least 10%, 30%, 50%, 75%, 150%, 500%, or 1,000%, or any value and range therebetween. Each possibility represents a separate embodiment of the invention. In some embodiments, increased binding affinity as used herein is by 1-10%, 5-30%, 20-50%, 35-75%, 70-150%, 100-500%, or 250-1,000%. Each possibility represents a separate embodiment of the invention. In some embodiments, increased binding affinity as used herein is by at least 1.5-fold, 2-fold, 5-fold, 10-fold, 50-fold, 100-fold, 500-fold, or 1,000-fold, or any value and range therebetween. Each possibility represents a separate embodiment of the invention.

Included within the scope of the invention are polypeptides or polypeptide fragments being at least 70%, 75%, 80%, 85%, 90%, or 95% identical to the microglia modulator described herein, or fragments thereof, or any value and range therebetween. Each possibility represents a separate embodiment of the invention.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology but is not necessarily translated from a designated nucleic acid sequence. It may be generated in any manner, including by chemical synthesis.

A polypeptide of the invention may be of a size of about 2 or more, 5 or more, 10 or more, 20 or more, 25 or more, 50 or more, 75 or more, 100 or more, 200 or more, 500 or more, 1,000 or more, or 2,000 or more amino acids, or any value and range therebetween. Each possibility represents a separate embodiment of the invention. Polypeptides may have a defined three-dimensional structure, although they do not necessarily have such structure. Polypeptides with a defined three-dimensional structure are referred to as folded, and polypeptides which do not possess a defined three-dimensional structure, but rather can adopt a large number of different conformations and are referred to as unfolded.

By an “isolated” polypeptide or a fragment, variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated for purpose of the invention, as are native or recombinant polypeptides which have been identified and separated, fractionated, or partially or substantially purified by any suitable technique.

In the present invention, a “polypeptide fragment” refers to a short amino acid sequence of a larger polypeptide. Protein fragments may be “free-standing,” or comprised within a larger polypeptide of which the fragment forms a part of region. Representative, non-limiting, examples of polypeptide fragments of the invention, include, for example, fragments comprising 5 amino acids, 10 amino acids, 15 amino acids, 20 amino acids, 30 amino acids, 40 amino acids, 50 amino acids, 60 amino acids, 70 amino acids, 80 amino acids, 90 amino acids, 100, 200, and 500 amino acids or more in length.

The terms “fragment,” “variant,” and “derivative” when referring to a polypeptide of the present invention include any polypeptide which retains at least some biological activity. Polypeptides as described herein may include fragment, variant, or derivative molecules without limitation, so long as the polypeptide still serves its function. Microglia modulators (e.g., anti-LAG-3 antibody) polypeptides and polypeptide fragments of the present invention may include proteolytic fragments, deletion fragments and in particular, fragments which more easily reach the site of action when delivered to an animal. Polypeptide fragments further include any portion of the polypeptide which comprises an antigenic or immunogenic epitope of the native polypeptide, including linear as well as three-dimensional epitopes. Polypeptides and polypeptide fragments of the present invention may comprise variant regions, including fragments as described above, and also polypeptides with altered amino acid sequences due to amino acid substitutions, deletions, or insertions.

Non-naturally occurring variants may be produced using art-known mutagenesis techniques. Polypeptides and polypeptide fragments of the invention may comprise conservative or non-conservative amino acid substitutions, deletions or additions and may also include derivative molecules. As used herein a “derivative” of a polypeptide or a polypeptide fragment refers to a subject polypeptide having one or more residues chemically derivatized by reaction of a functional side group. Also included as “derivatives” are those peptides which contain one or more naturally occurring amino acid derivatives of the twenty standard amino acids. For example, 4-hydroxyproline may be substituted for proline; 5-hydroxylysine may be substituted for lysine; 3-methylhistidine may be substituted for histidine; homoserine may be substituted for serine; and ornithine may be substituted for lysine.

In some embodiments, a polypeptide of the invention is an antibody. The term “antibody” is used in the broadest sense and specifically encompasses polyclonal and monoclonal antibodies and antibody fragments so long as they exhibit the desired biological activity. In some embodiments, the use of a chimeric antibody or a humanized antibody, derivative or fragment thereof, is also encompassed by the invention. In some embodiments, an antibody is a neutralizing antibody.

In some embodiments, an antibody derivative or fragment thereof comprises a portion of an intact antibody, comprising the antigen binding region thereof. Examples of antibody fragments include Fab, Fab′, F(ab′)2, and Fv fragments; diabodies; tandem diabodies (taDb), linear antibodies (e.g., U.S. Pat. No. 5,641,870, Example 2; Zapata et al, Protein Eng. 8(10): 1057-1062 (1995)); one-armed antibodies, single variable domain antibodies, minibodies, single-chain antibody molecules; multi-specific antibodies formed from antibody fragments (e.g., including but not limited to, Db-Fc, taDb-Fc, taDb-CH3, (scFV)4-Fc, di-scFv, bi-scFv, or tandem (di, tri)-scFv); and Bi-specific T-cell engagers (BiTEs). In some embodiment, an antibody derivative or fragment thereof, includes a Fc.

In some embodiments, the antibody or fragment thereof is a part of a bispecific antibody that can facilitate the penetration of the microglia-modulating antibody via the blood-brain-barrier (BBB), bringing it to contact with microglia. In some embodiments, the bispecific antibody comprises: (1) an inhibitory compound which binds for example to LAG-3, CD180, TDO2, Cd86/B7-2, PD-L1, or PLA2G4E, and (2) a molecule enabling receptor-mediated transcytosis across the BBB. In some embodiments, the molecule enabling receptor-mediated transcytosis across the BBB can be represented as a part of the bispecific antibody, and is selected from: transferrin receptor, insulin receptor (InsR), Lrp1, Lrp2, TMEM 30A, heparin-binding epidermal growth factor-like growth factor (HB-EGF), basigin, Glut1, or CD98hc.

In some embodiments, Fv is the minimum antibody fragment that contains a complete antigen-recognition and antigen-binding site. In some embodiments, a Fv derivative or fragment thereof, comprising only three hypervariable regions specific for an antigen, has the ability to recognize and bind antigen. In one embodiment, Fv has a higher binding affinity to an antigen compared to a Fv derivative or fragment thereof.

In some embodiments, the term “diabodies” refer to small antibody fragments with two antigen-binding sites.

In some embodiments, non-human antibodies may be humanized by any methods known in the art. In one method, the non-human complementarity determining regions (CDRs) are inserted into a human antibody or consensus antibody framework sequence. Further changes can then be introduced into the antibody framework to modulate affinity or immunogenicity.

In some embodiments, neutralizing antibodies include: antibodies, fragments of antibodies, Fab and F(ab′)2, single-domain antigen-binding recombinant fragments and nanobodies.

The term “polynucleotide” is intended to encompass a singular nucleic acid as well as plural nucleic acids and refers to an isolated nucleic acid molecule or construct, e.g., messenger RNA (mRNA) or plasmid DNA (pDNA). A polynucleotide can contain the nucleotide sequence of the full-length cDNA sequence, including the untranslated 5′ and 3′ sequences, the coding sequences. A polynucleotide may comprise a conventional phosphodiester bond or a non-conventional bond (e.g., an amide bond, such as found in peptide nucleic acids (PNA)). The polynucleotide can be composed of any polyribonucleotide or polydeoxyribonucleotide, which may be unmodified RNA or DNA or modified RNA or DNA. For example, polynucleotides can be composed of single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, the polynucleotides can be composed of triple-stranded regions comprising RNA or DNA or both RNA and DNA. Polynucleotides may also contain one or more modified bases, DNA or RNA backbones modified for stability, or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

The term “nucleic acid” refers to any one or more nucleic acid segments, e.g., DNA or RNA fragments, present in a polynucleotide. By “isolated” nucleic acid or polynucleotide is intended a nucleic acid molecule, DNA or RNA, which has been removed from its native environment. For example, anti-LAG-3 antibody contained in a vector is considered isolated for the purposes of the present invention. Further examples of an isolated polynucleotide include recombinant polynucleotides maintained in heterologous host cells or purified (partially or substantially) polynucleotides in solution. Isolated RNA molecules include in vivo or in vitro RNA transcripts of polynucleotides of the present invention. Isolated polynucleotides or nucleic acids according to the present invention further include such molecules produced synthetically. In addition, a polynucleotide or nucleic acid may be or may include a regulatory element such as a promoter, ribosome binding site, or a transcription terminator.

In some embodiments, a microglia modulator of the present invention may be administered and/or used in a composition with a second immune/microglia stimulator. Non-limiting examples of immune/microglia stimulators are selected from the group consisting of: M-CSF; GM-CSF; IL-34; G-CSF; soluble LAG-3 and CX3CR1 blockers. As used herein, “blockers” refer to any molecule capable of binding yet preventing signal propagation, such as antagonists and blocking antibodies. Non-limiting examples include: agonist (activating) antibodies to CD137, a member of the tumor necrosis factor (TNF) receptor family; agonist (activating) antibodies to glucocorticoid-induced tumor necrosis factor receptor family-related protein (GITR); agonist (activating) antibodies to OX40 (tumor necrosis factor receptor superfamily, member 4); inhibitors of indoleamine 2,3-dioxygenase-1 (IDO1); CD40 ligand (CD154); Interferon gamma (IFNγ); Monophosphoryl lipid A (MPL); Protollin; Amphotericin B (AmB) (Fungizone); polyinosinic-polycytidylic acid (poly (I:C); CpG oligonucleotides; aluminum hydroxide (alum); MF59; Adjuvant System 03 (AS03); imiquimod; loxoribine; R-848; 12-myristate 13-acetate (PMA); Lipopolysaccharide (LPS); Endotheline; a-Crystallin (small heat shock protein); Platelet-activating factor (PAF); anti-ICOS; anti-B7RP1; anti-VISTA; anti-CD40; anti-CD40L; anti-CD80; anti-CD86; anti-B7-H3; anti-B7-H4; CD anti-B7-H7; anti-BTLA; anti-HVEM; anti-CD 137; anti-CD 137L; anti-OX40L; anti-CD-27; anti-CD70; anti-STING; anti-TIGIT; anti-PD-1 (CD 279); anti-CTLA-4 (CD152); anti-CD200R1, an adenosine A1 receptor antagonist; an adenosine A2a receptor antagonist; and any combination thereof.

### Treatment

In some embodiments, the method of the invention is directed to treating a depression disorder in an antidepressant-non-treated subject. In some embodiments, the antidepressant is selected from: Selective serotonin reuptake inhibitors (SSRIs), Serotonin norepinephrine reuptake inhibitors (SNRIs), Serotonin-dopamine reuptake inhibitors (SDRIs), Norepinephrine-dopamine reuptake inhibitors (NDRIs), Serotonin antagonist and reuptake inhibitors (SARIs), Tricyclic antidepressants (TCAs), Tetracyclic antidepressants (TeCAs), Noradrenergic and specific serotonergic antidepressants (NaSSAs), Monoamine oxidase inhibitors (MAOIs), Reversible inhibitors of monoamine oxidase A (RIMAs), NMDA receptor antagonists (NMDARAs), and any combination thereof.

In some embodiments, the antidepressant is a SSRI.

Is some embodiments, the subject in a SSRI non-treated subject.

Types of SSRI would be apparent to one of ordinary skill in the art. Non-limiting examples include, but are not limited to, Escitalopram, Citalopram, Fluoxetine, Fluvoxamine, Paroxetine, Sertraline, and others.

In some embodiment, the present invention is directed to methods for treating or attenuating a depressive disorder in a subject having normal or low inflammatory state.

In another embodiment, inflammatory state is detected by determining the level of activated microglia. In another embodiment, inflammatory state is detected by determining the level of dystrophic microglia. In another embodiment, low inflammatory state is an increase in dystrophic microglia.

In some embodiments, normal or low inflammatory state is detected by determining the level of at least one inflammatory marker. In another embodiment, said inflammatory marker is C-reactive protein (CRP). In some embodiments, CRP is a sensitive, nonspecific, acute-phase protein, produced in response to most forms of tissue injury, infection, and inflammation. In some embodiments, CRP is produced by Kupffer cells in the liver, which are regulated by cytokines, such as IL-1, IL-6 and TNFα. Based on its stability, assay precision, accuracy, and availability; and the availability of standards for proper assay calibration, the high sensitivity CRP assay was recommended as the preferred inflammatory marker for coronary vascular disease. In some embodiments, in normal humans, with no overt inflammatory condition, 95% of the population has CRP values lower than 10 mg/L. In another study more than 50% of the normal population was found to have CRP levels lower than 2 mg/L (Koenig et al., 1999).

In another embodiment, additional inflammatory markers can be utilized for detection of a low inflammatory state, including IL-6 and TNFα. In some embodiments, methods of the present invention are directed to treatment of a subject suffering from a depression condition or disorder having IL-6 or TNFα levels lower than the levels of these cytokines in a control population (i.e., not having an inflammatory disease or disorder), typically less than 2.0 pg/ml for IL-6 and 3.8 pg/ml for TNFα. In some embodiments, Erythrocyte Sedimentation Rate (ESR) can also be used to define the inflammatory state. In some embodiments, methods of the present invention are directed to treatment of a subject suffering from a depression condition or disorder having less than 6.3 mm/h for ESR.

In another embodiment, inflammatory state (e.g., levels of activated or dystrophic microglia) is assessed by positron emission tomography (PET) imaging. As known to one skilled in the art, microglia express the 18 kDa translocator protein (TSPO), which can be quantified by several PET ligands (Owen and Matthews, Int Rev Neurobiol. 2011; 101:19-39). The most common ligand is [(11)C]PK11195 (also termed peripheral benzodiazepine receptor), but newer ligand, such as [18F]-FEPPA, [11C]PBR28 and [18F]DPA are also available.

In some embodiments, the methods of the invention comprise assessing the inflammatory status of a subject at least twice, such as before and after treatment. In some embodiments, a subject is treated with a microglia modulator of the invention when the microglia levels or activation status are determined to be low or decreasing.

In another embodiment, low inflammatory state is assessed by comparing the inflammatory state of a subject to a pre-determined inflammatory level. In another embodiment, pre-determined inflammatory level is a pre-determined control level. In another embodiment, pre-determined inflammatory level is an inflammatory level previously detected in the subject.

According to some embodiments, the present invention is directed to treating a subject having altered transcript levels of one or more transcripts selected from the group consisting of: lymphocyte activating gene 3 (Lag-3), Cluster of differentiation molecule 180 (Cd-180), tryptophan 2,3-dioxygenase (Tdo2), Colony stimulating factor 2 receptor beta common subunit (Csf2rb2), Major histocompatibility complex, class I, A (H2-d1), Zinc finger CCHC-type containing 5 (Zcchc5), MafbZIP transcription factor A (MafA), Phospholipase A2 group IVE (Pla2g4e), SRY-box 11 (Sox11), Synaptic vesicle glycoprotein 2C (Sv2c), Dopamine receptor D1 (Drd1), Protein tyrosine phosphatase, receptor type, V (Ptprv), Protein disulfide isomerase family A member 4 (Pdia4), Serine incorporator 2 (Serinc2) and NADP dependent oxidoreductase domain containing 1 (Noxred1), compared to a baseline level.

According to some embodiments, the present invention is directed to treating a subject having increased transcript levels of one or more transcripts selected from the group consisting of: Lag-3, Cd-180, Tdo2, Csf2rb2, H2-dl, Zcchc5, MafA and Pla2g4e compared to a baseline level in a sample derived from the subject.

According to some embodiments, the present invention is directed to treating a subject having decreased transcript levels of one or more transcripts selected from the group consisting of: Sox11, Sv2c, Drd1, Ptprv, Pdia4, Serinc2 and Noxred1 compared to a baseline level.

According to some embodiments, the present invention is directed to treating a subject having increased transcript levels of Lag-3, Cd-180, Tdo2, Csf2rb2, H2-dl, Zcchc5, MafA and Pla2g4e; and decreased transcript levels of Sox11, Sv2c, Drd1, Ptprv, Pdia4, Serinc2 and Noxred1 compared to a baseline level in a sample derived from the subject.

According to some embodiments, the present invention is directed to a method of treating a subject having increased transcript levels of any one of: Lag-3, Cd-180, Tdo2, Csf2rb2, H2-d1, Zcchc5, MafA, or Pla2g4e; or decreased transcript levels of any one of: Sox11, Sv2c, Drd1, Ptprv, Pdia4, Serinc2, or Noxred1.

According to some embodiments, the present invention is directed to a method of treating a subject having increased transcript levels of any one of: Lag-3, Cd-180, Tdo2, Csf2rb2, H2-dl, Zcchc5, MafA, or Pla2g4e; and decreased transcript levels of any one of: Sox11, Sv2c, Drd1, Ptprv, Pdia4, Serinc2, or Noxred1.

In some embodiments, the aforementioned increased or decreased transcript levels are detected in sample derived from or obtained from the subject.

In some embodiments, the sample comprises bodily fluid. In some embodiments, the sample comprises a cell. In some embodiments, the sample comprises a tissue or a fragment thereof. In some embodiments, the sample comprises whole blood. In some embodiments, the sample comprises peripheral blood mononuclear cells (PBMCs). In some embodiments, the sample comprises isolated T cells. In some embodiments, the sample comprises isolated dendritic cells. In some embodiments, the sample comprises isolated monocytes.

Any one of the following transcripts: Lag-3, Cd-180, Tdo2, Csf2rb2, H2-d1, Zcchc5, MafA and Pla2g4e, Sox11, Sv2c, Drd1, Ptprv, Pdia4, Serinc2 and Noxred1, is referred to as “a specific transcript” herein below.

In some embodiments, a subject is pre-selected for treatment based on one or more expression levels of specific transcripts. According to some embodiments, the methods of the present invention comprise a step of selecting a subject having altered transcript levels of one or more transcripts selected from the group consisting of: Lag-3, Cd-180, Tdo2, Csf2rb2, H2-dl, Zcchc5, MafA and Pla2g4e, Sox11, Sv2c, Drd1, Ptprv, Pdia4, Serinc2 and Noxred1, compared to a baseline level.

In some embodiments, the specific transcripts expression levels are altered. In some embodiments, alterations comprise over-expression of specific transcripts. In some embodiments, alterations comprise reduction of specific transcripts. In some embodiments, alteration comprise over-expression of specific transcripts and reduction of other transcripts.

In some embodiments, alterations of transcript levels are in comparison to a baseline level. As defined herein, the term “baseline level” refers to the level of a specific transcript measured in the subject before or at early symptoms of a condition. In another embodiment, an altered level of a specific transcript in a subject is measured compared to any other tissue in the subject but microglia. In one embodiment, an altered level of any specific transcript in a subject is measured compared to a non-afflicted control subject.

As used herein, the terms “increased transcript level” and “over-expression” are interchangeable. In one embodiment, increased transcript level is by at least 10%, 30%, 50%, 75%, 100%, 150%, 250%, 500% or 1,000% compared to a baseline level. In one embodiment, increased transcript level as used herein is by 1-10%, 5-30%, 20-50%, 35-75%, 40-100%, 60-150%, 110-250%, 220-500%, or 350-1,000% compared to a baseline level, or any value and range therebetween. Each possibility represents a separate embodiment of the invention. In one embodiment, increased transcript level as used herein is by at least 1.5-fold, 2-fold, 5-fold, 10-fold, 50-fold, 100-fold, 500-fold, or 1,000-fold compared to a baseline level, or any value and range therebetween. Each possibility represents a separate embodiment of the invention.

As used herein, the term “reduction of specific transcripts” refers to decrease in number of a specific gene's mRNA molecules. In one embodiment, reduced transcript levels as used herein is by at least 10%, 30%, 50%, 75%, 100%, 150%, 250%, 500%, or 1,000% compared to a baseline level, or any value and range therebetween. Each possibility represents a separate embodiment of the invention. In one embodiment, reduced transcript as used herein is by 1-10%, 5-30%, 20-50%, 35-75%, 40-100%, 60-150%, 110-250%, 220-500%, or 350-1,000% compared to a baseline level. Each possibility represents a separate embodiment of the invention. In one embodiment, reduced transcript as used herein is by at least 1.5-fold, 2-fold, 5-fold, 10-fold, 50-fold, 100-fold, 500-fold, or 1,000-fold compared to a baseline level, or any value and range therebetween. Each possibility represents a separate embodiment of the invention.

Numerous methods are known in the art for measuring expression levels of a one or more gene such as by amplification of nucleic acids (e.g., PCR, isothermal methods, rolling circle methods, etc.) or by quantitative in situ hybridization. Design of primers for amplification of specific genes is well known in the art, and such primers can be found or designed on various websites such as http://bioinfo.ut.ee/primer3-0.4.0/ or https://pga.mgh.harvard.edu/primerbank/ for example.

The skilled artisan will understand that these methods may be used alone or combined. Non-limiting exemplary method are described herein.

RT-qPCR: A common technology used for measuring RNA abundance is RT-qPCR where reverse transcription (RT) is followed by real-time quantitative PCR (qPCR). Reverse transcription first generates a DNA template from the RNA. This single-stranded template is called cDNA. The cDNA template is then amplified in the quantitative step, during which the fluorescence emitted by labeled hybridization probes or intercalating dyes changes as the DNA amplification process progresses. Quantitative PCR produces a measurement of an increase or decrease in copies of the original RNA and has been used to attempt to define changes of gene expression in cancer tissue as compared to comparable healthy tissues.

RNA-Seq: RNA-Seq uses recently developed deep-sequencing technologies. In general, a population of RNA (total or fractionated, such as poly(A)+) is converted to a library of cDNA fragments with adaptors attached to one or both ends. Each molecule, with or without amplification, is then sequenced in a high-throughput manner to obtain short sequences from one end (single-end sequencing) or both ends (pair-end sequencing). The reads are typically 30-400 bp, depending on the DNA-sequencing technology used. In principle, any high-throughput sequencing technology can be used for RNA-Seq. Following sequencing, the resulting reads are either aligned to a reference genome or reference transcripts or assembled de novo without the genomic sequence to produce a genome-scale transcription map that consists of both the transcriptional structure and/or level of expression for each gene. To avoid artifacts and biases generated by reverse transcription direct RNA sequencing can also be applied.

Microarray: Expression levels of a gene may be assessed using the microarray technique. In this method, polynucleotide sequences of interest (including cDNAs and oligonucleotides) are arrayed on a substrate. The arrayed sequences are then contacted under conditions suitable for specific hybridization with detectably labeled cDNA generated from RNA of a test sample. As in the RT-PCR method, the source of RNA typically is total RNA isolated from a tumor sample, and optionally from normal tissue of the same patient as an internal control or cell lines. RNA can be extracted, for example, from frozen or archived paraffin-embedded and fixed (e.g., formalin-fixed) tissue samples. For archived, formalin-fixed tissue cDNA-mediated annealing, selection, extension, and ligation, DASL-Illumina method may be used. For a non-limiting example, PCR amplified cDNAs to be assayed are applied to a substrate in a dense array. Microarray analysis can be performed.

In some embodiments, the microglia modulator, for example, a compound blocking the binding of MHC II to a LAG-3 receptor is administered once per day, continuously or intermittently, such as until there is an improved in said mood or depressive symptomatology.

In some embodiments, the therapeutically effective amount of the microglia modulator, for example, a compound blocking the binding of MHC II to a LAG-3 receptor is from between 0.1 and 100 μg/kg body weight per day, 1 and 100 μg/kg body weight per day, 1 and 75 μg/kg body weight per day, 1 and 50 μg/kg body weight per day, 1 and 40 μg/kg body weight per day, 1 and about 30 μg/kg body weight per day, or 1 and 25 μg/kg body weight per day. Each possibility represents a separate embodiment of the invention.

In some embodiments, soluble LAG-3 compound used as anon-limiting example for a microglia modulator, is administered once per day, continuously or intermittently, such as until there is an improved in said mood or depressive symptomatology.

In some embodiments, the therapeutically effective amount of the soluble LAG-3 compound used as a non-limiting example for a microglia modulator, is from between 0.1 and 100 μg/kg body weight per day, 1 and 100 μg/kg body weight per day, 1 and 75 μg/kg body weight per day, 1 and 50 μg/kg body weight per day, 1 and 40 μg/kg body weight per day, 1 and about 30 μg/kg body weight per day, or 1 and 25 μg/kg body weight per day. Each possibility represents a separate embodiment of the invention.

In some embodiments, microglia modulator of the present invention is administered to the subject at least once per day. In one embodiment, microglia modulator of the present invention is administered to the subject on alternating days. In one embodiment, microglia modulator of the present invention is administered to the subject at least once every 3 days. In one embodiment, microglia modulator of the present invention is administered to the subject at least once every 7 days. In one embodiment, microglia modulator of the present invention is administered to the subject at least once per week. In one embodiment, microglia modulator of the present invention is administered to the subject at least twice a week. In one embodiment, microglia modulator of the present invention is administered to the subject at least once per two weeks.

As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, or provide improvement to a patient or subject's quality of life.

By “subject” or “individual” or “animal” or “patient” or “mammal,” is meant any subject, particularly a mammalian subject, for whom diagnosis, prognosis, or therapy is desired. Mammalian subjects include, but are not limited to, humans, domestic animals, farm animals, zoo animals, sport animals, pet animals such as dogs, cats, guinea pigs, rabbits, rats, mice, horses, cattle, cows; primates such as apes, monkeys, orangutans, and chimpanzees; canids such as dogs and wolves; felids such as cats, lions, and tigers; equids such as horses, donkeys, and zebras; food animals such as cows, pigs, and sheep; ungulates such as deer and giraffes; rodents such as mice, rats, hamsters and guinea pigs; and so on. In certain embodiments, the mammal is a human subject.

The term “depression condition or disorder” includes but is not limited to, depression of any type, including but not limited to unipolar major depressive episode, major depressive disorder, dysthymic disorder, treatment-resistant depression, bipolar depression, adjustment disorder with depressed mood, cyclothymic disorder, melancholic depression, psychotic depression, post-schizophrenic depression, depression due to a general medical condition, as well as to post-viral fatigue syndrome, and chronic fatigue syndrome. In one embodiment, depression is a stress-induced depression.

In some embodiments, the invention includes treatment of a subject afflicted by schizophrenia, and particularly a schizophrenic subject characterized by low number and activity of microglia. In some embodiments, said subject is a schizophrenic subject afflicted by depression. In some embodiments said subject shows schizophrenic symptoms including but not limited to anhedonia and/or apathy, and/or social problems/withdrawal. In some embodiments, the invention includes treatment of subtypes of schizophrenia, including but not limited to paranoid schizophrenia, disorganized schizophrenia, catatonic schizophrenia, undifferentiated schizophrenia, residual schizophrenia and simple schizophrenia.

The term “stress-induced condition or disorder” includes but is not limited to stress-related disorders, including but not limited to Posttraumatic Stress Disorder, Acute Stress Disorder, Adjustment Disorder, Bereavement Related Disorder, Other Specified Trauma- or Stressor-Related Disorder and Unspecified Trauma, Generalized Anxiety Disorder, Anxiety Disorder due to general medical condition, and Anxiety disorder not otherwise specified.

In some embodiments, a stress-induced condition or disorder is a chronic state. In some embodiments, a stress-induced condition or disorder is an acute state. In some embodiments, a stress-induced condition encompasses secretion of corticosteroids, and catecholamines (e.g., epinephrine and norepinephrine). All methods of detection and quantification of corticosteroids and catecholamines are acceptable and would be known to one of ordinary skill in the art. Non-limiting examples include ELISA and mass spectrometry (such as LC-MS-MS).

In some embodiments, the treatment is sufficient in improving at least one parameter related to depression and/or stress responsiveness, including, but not limited to, depressed mood, anhedonia, decrease in appetite and significant weight loss, insomnia or hypersomnia, psychomotor retardation, fatigue or loss of energy, diminished ability to think or concentrate or indecisiveness, helplessness, hopefulness, recurrent thoughts of death, a suicide attempt or a specific plan for committing suicide, excessive anxiety, uncontrollable worry, restlessness or feeling keyed up or on edge, being easily fatigued, difficulty concentrating or mind going blank, irritability, sleep disturbance (difficulty falling or staying asleep, or restless unsatisfying sleep), sense of numbing, detachment, or absence of emotional responsiveness, a reduction in awareness of his or her surroundings, depersonalization, derealization, anxiety or increased arousal (e.g., difficulty sleeping, irritability, poor concentration, hypervigilance, exaggerated startle response, motor restlessness), avoidance of places and situations, distress or impairment in social, occupational, or other important areas of functioning.

In another embodiment, the method further comprises administering to the subject at least one of the following anti-depressant drugs, including fluoxetine, sertraline, venlafaxine, citalopram, parocetine, trazodone, amitriptyline, nortriptyline, imipramine, dothiepin, lofepramine, doxepin, protriptyline, tranylcypromine, moclobemide, bupropion, nefazodone, mirtazapine, zolpidem, alprazolam, temazepam, diazepam, or buspirone.

### Non-Invasive Brain Stimulation (NIBS)

In some embodiments, the methods of the present invention are directed to treating the subject by a non-invasive brain stimulation (NIBS).

As used herein, the term “NIBS” refers to any stimulation technique aiming to alter brain activity by induction of an electrical, and/or magnetic stimulation of the brain.

In some embodiments, NIBS is applied in cases of severe depression. In some embodiments, severe depression encompasses psychosis, suicidal behavior or refusal to eat. In some embodiments, NIBS is applied in cases of treatment-resistant depression. In one embodiment, treatment-resistance is a case in which no improvement with either medication or other treatment is observed. In some embodiments, NIBS is applied in cases of severe mania. In some embodiments, severe mania encompasses intense euphoria, agitation, hyperactivity, impaired decision-making, impulsive behavior, substance abuse and psychosis. In some embodiments, NIBS is applied in cases of catatonia. In some embodiments, catatonia encompasses lack of or irregular movements, lack of speech, or others. In some embodiments, catatonia is associated with schizophrenia and other psychiatric disorders. In one embodiment, catatonia is a result of a medical illness. In some embodiments, NIBS is applied in cases of agitation and aggression associated with dementia.

In some embodiments, NIBS is applied in cases where standard medications or other form of therapy are not tolerated or cannot be administrated and include, but not limited to, pregnancy (induction abnormal fetal development) and treating the elderly (intolerable side effects). In some embodiments, NIBS is applied when a subject chooses NIBS over taking medications. In some embodiments, NIBS is re-applied in cases where it has been successfully applied in the past.

In some embodiments, the methods of the present invention comprise a combined treatment, comprising administration of microglia modulator(s) and application of NIBS, such as ECT.

An individual treated by the methods of the present invention who exhibits an “increased therapeutic response to NIBS” may be placed on a modified NIBS treatment schedule that consists of fewer, less frequent, or shorter NIBS treatments. A modification of NIBS treatment includes any modification that would render NIBS safer to administer to an individual including, for example, a reduction in the electrical intensity, magnetic intensity, or stimulus dosage of the NIBS.

In some embodiments, the method provides reducing the frequency and/or duration of NIBS. As used herein, reducing the frequency and/or duration is a reduction of at least 5%, at least 10%, at least 15%, at least 30%, at least 50%, at least 75%, or at least 100%, of NIBS frequency and/or duration, as would be applied without the administration of a microglia modulator, or any value and range therebetween. Each possibility represents a separate embodiment of the invention. In some embodiments, reducing the frequency and/or duration is a reduction of 5-15%, 10-30%, 20-50%, 40-75%, or 65-100%, of NIBS frequency and/or duration, as would be applied without the administration of a microglia modulator. Each possibility represents a separate embodiment of the invention.

In some embodiments, the ratio between microglia modulator(s) administration events and NIBS application events is 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, or 1:10, or any range therebetween. Each possibility represents a separate embodiment of the invention.

In some embodiments, every event of NIBS application is followed by at least 1, 2, 3, 4, 5, 6, 7, 8, 9, or 10 events of microglia modulator(s) administration, or any value and range therebetween. Each possibility represents a separate embodiment of the invention. In some embodiments, every event of NIBS application is followed by 1-2, 1-3, 2-4, 3-5, 4-6, 5-7, 6-8, 7-9, or 6-10 events of microglia modulator(s) administration. Each possibility represents a separate embodiment of the invention. In some embodiments, every event of microglia modulator(s) administration is followed by at least 1, 2, 3, 4, 5, 6, 7, 8, 9, or 10 events of NIBS application, or any value and range therebetween. Each possibility represents a separate embodiment of the invention. In some embodiments, every event of microglia modulator(s) administration is followed by 1-2, 1-3, 2-4, 3-5, 4-6, 5-7, 6-8, 7-9, or 6-10 events of NIBS application. Each possibility represents a separate embodiment of the invention. In one embodiment, NIBS is applied twice a week. In one embodiment, NIBS is applied three times a week. In some embodiments, NIBS is applied over a course of 3 weeks. In some embodiments, NIBS is applied over a course of 4 weeks. In some embodiments NIBS application comprises a total of 6 to 12 treatments after which subject is recovered for a period of at least 2, 3, 4, 5, 6, 7, 8, 9, 10, or 11 months, or any value and range therebetween. Each possibility represents a separate embodiment of the invention. In one embodiment, during recovery period subject is treatment free.

In some embodiments, the methods of the present invention provide administration of microglia modulator(s) as a therapy for replacing NIBS. In some embodiment, the treatment methods of the present invention do not comprise NIBS.

In some embodiments, there is provide a method of increasing the therapeutic response to NIBS.

The term “increasing the therapeutic response to NIBS” refers to an indicium of success in NIBS treatment of a disease amenable to NIBS, including any objective or subjective parameter such as abatement, remission or diminishing of symptoms or an improvement in a patient's physical or mental well-being. Amelioration of symptoms can be based on objective or subjective parameters: including the results of a physical examination and/or a psychiatric evaluation. For example, a clinical guide to monitor the effective amelioration of a mental disorder, such as psychotic major depression or melancholic depression, is found in the Structured Clinical Interview for DSM-IV Axis I mood disorders (“SCID-P”).

In some embodiments, there is provide a method of decreasing the severity or occurrence of side effects typically associated with NIBS. Side effects associated with NIBS include any negative effect that is a by-product of the NIBS treatment. Negative side effects, for example, may include tachycardia, atrial arrhythmia, ventricular arrhythmia, hypertension, asystole, muscle pain, fatigue, headaches, nausea, amnesia, and confusion.

In some embodiments, NIBS comprises a method selected from: repetitive transcranial magnetic stimulation (rTMS), deep TMS, cranial electrotherapy stimulation (CES), transcranial direct current stimulation (tDCS), transcranial random noise stimulation (tRNS), reduced impedance non-invasive cortical electrostimulation (RINCE), electroconvulsive therapy (ECT), or a combination thereof.

As used herein, the term “rTMS” encompasses the use of external magnetic field pulses.

As used herein, the term “tDCS” encompasses the use of mild electrical current. In one embodiment, the term “mild” is compared to ECT. In one embodiment, electrical currents applied by means of ECT are stronger than the currents applied by means of tDCS.

As used herein, “ECT” refers to small electric currents that are transmitted to the brain, intentionally to trigger a brief seizure.

In some embodiments, ECT comprises unilaterally or bilaterally applied ECT. In one embodiment, unilaterally ECT is applied by a right unilateral ultra-brief pulse.

As used herein, the term “ECT eligibility” encompasses all cases not applying as ECT ineligibility. In some embodiments, a subject ineligible for ECT application is a having unstable or severe cardiovascular conditions, aneurysm or vascular malformation, increased intracranial pressure, cerebral infarction, pulmonary insufficiency and medical status rated by the American Society of Anesthesiologists (ASA) as level 4 or 5. In some embodiments, an ECT eligible subject encompasses subjects with coexisting medical illness, as well as the elderly, pregnant women, nursing mothers, children and young adults. In some embodiments, reducing risks of ECT can be achieved by changing the subject's preparation, adjusting treatment's delivery methods, and any other approach known to one of ordinary skill in the field of ECT.

In one embodiment, a NIBS method utilized according to the present invention is ECT.

### Pharmaceutical Compositions

According to some embodiments, the present invention provides a pharmaceutical composition for use in treating a depression condition or disorder in a subject, the pharmaceutical composition comprising a therapeutically effective amount of at least one microglia modulator selected from compounds blocking LAG-3, Cd180, TDO2, B7-2, PD-L1, PLA2G4E, or an active variant, fragment or derivative thereof, or any combination thereof, and at least one pharmaceutically acceptable carrier or diluent.

In some embodiments, the composition comprises an antibody or fragment thereof. In some embodiments, the composition comprises a bispecific antibody having BBB penetration capabilities. In some embodiments, the composition comprises a bispecific antibody comprising: an inhibitory compound which binds for example to LAG-3, CD180, TDO2, Cd86/B7-2, PD-L1, or PLA2G4E, and (2) a molecule enabling receptor-mediated transcytosis across the BBB. In some embodiments, the composition comprises a molecule enabling receptor-mediated transcytosis across the BBB. In some embodiments, the composition comprises a molecule having specific binding affinity to: transferrin receptor, insulin receptor (InsR), Lrp1, Lrp2, TMEM 30A, heparin-binding epidermal growth factor-like growth factor (HB-EGF), basigin, Glut1, or CD98hc.

The term “pharmaceutical composition”, as used herein, refers to at least one microglia modulator with chemical components such as diluents or carriers that do not cause unacceptable adverse side effects and that do not prevent microglial modulation.

As used herein, a “therapeutically effective amount” or “an amount effective” refers to an amount effective, at dosages and for periods of time necessary, to achieve the desired therapeutic result. A therapeutic result may be, e.g., lessening of symptoms, prolonged survival, improved mobility, improved social and vocational functioning, and the like. A therapeutic result need not be a “cure.” A therapeutic result may also be prophylactic. Typically, since a prophylactic dose is used in subjects prior to or at an earlier stage of disease, the prophylactically effective amount will be less than the therapeutically effective amount.

The amount of the peptides of the present invention, which will be effective in the treatment of a particular disorder or condition will depend on the nature of the disorder or condition and on the particular peptide and can be determined by standard clinical techniques known to a person skilled in the art. In addition, in vitro assays may optionally be employed to help identify optimal dosage ranges. The precise dose to be employed in the formulation will also depend on the route of administration, and the nature of the disease or disorder, and should be decided according to the judgment of the practitioner and each patient's circumstances. Effective doses can be extrapolated from dose-response curves derived from in-vitro or in-vivo animal model test bioassays or systems.

The pharmaceutical compositions of the invention can be formulated in the form of a pharmaceutically acceptable salt of the peptides of the present invention or their analogs, or derivatives thereof. Pharmaceutically acceptable salts include those salts formed with free amino groups such as salts derived from non-toxic inorganic or organic acids such as hydrochloric, phosphoric, acetic, oxalic, tartaric acids, and the like, and those salts formed with free carboxyl groups such as salts derived from non-toxic inorganic or organic bases such as sodium, potassium, ammonium, calcium, ferric hydroxides, isopropylamine, triethylamine, 2-ethylamino ethanol, histidine, procaine, and the like.

The term “pharmaceutically acceptable” means suitable for administration to a subject, e.g., a human. For example, the term “pharmaceutically acceptable” can mean approved by a regulatory agency of the Federal or a state government or listed in the U. S. Pharmacopeia or other generally recognized pharmacopeia for use in animals, and more particularly in humans. The term “carrier” refers to a diluent, adjuvant, excipient, or vehicle with which the therapeutic compound is administered. Such pharmaceutical carriers can be sterile liquids, such as water and oils, including those of petroleum, animal, vegetable or synthetic origin, such as peanut oil, soybean oil, mineral oil, sesame oil and the like, polyethylene glycols, glycerin, propylene glycol or other synthetic solvents. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like. The composition, if desired, can also contain minor amounts of wetting or emulsifying agents, or pH buffering agents such as acetates, citrates or phosphates. Antibacterial agents such as benzyl alcohol or methyl parabens; antioxidants such as ascorbic acid or sodium bisulfite; and agents for the adjustment of tonicity such as sodium chloride or dextrose are also envisioned.

The compositions can take the form of solutions, suspensions, emulsions, colloidal dispersions, emulsions (oil-in-water or water-in-oil), sprays, aerosol, ointment, tablets, pills, capsules, powders, gels, creams, ointments, foams, pastes, sustained-release formulations and the like. In particular embodiments, the pharmaceutical compositions of the present invention are formulated for aerosol administration for inhalation by a subject in need thereof.

In some embodiments, the composition of the invention is administered by intranasal or intraoral administration, using appropriate solutions, such as nasal solutions or sprays, aerosols or inhalants. Nasal solutions are usually aqueous solutions designed to be administered to the nasal passages in drops or sprays. Typically, nasal solutions are prepared so that they are similar in many respects to nasal secretions. Thus, the aqueous nasal solutions usually are isotonic and slightly buffered to maintain a pH of 5.5 to 6.5. In addition, antimicrobial preservatives, similar to those used in ophthalmic preparations, and appropriate drug stabilizers, if required, may be included in the formulation. Various commercial nasal and oral preparations for inhalation, aerosols and sprays are known and include, for example, antibiotics and antihistamines and are used for asthma prophylaxis.

For intranasal or intraoral administration, the composition of the invention is provided in a solution suitable for expelling the pharmaceutical dose in the form of a spray, wherein a therapeutic quantity of the pharmaceutical composition is contained within a reservoir of an apparatus for nasal or intraoral administration. The apparatus may comprise a pump spray device in which the means for expelling a dose comprises a metering pump. Alternatively, the apparatus comprises a pressurized spray device, in which the means for expelling a dose comprises a metering valve and the pharmaceutical composition further comprises a conventional propellant. Suitable propellants include one or mixture of chlorofluorocarbons, such as dichlorodifiuoromethane, trichlorofiuoromethane, dichloro-tetrafluoroethane, hydrofluorocarbons, such as 1,1,1,2-tetrafiuoroethane (HFC-134a) and 1,1,1,2,3,3,3-heptafluoropropane (HFC-227) or carbon dioxide. Suitable pressurized spray devices are well known in the art and include those disclosed in, inter alia, WO 92/11190, U.S. Pat. Nos. 4,819,834, 4,407,481 and WO 97/09034, when adapted for producing a nasal spray, rather than an aerosol for inhalation, or a sublingual spray. The contents of the aforementioned publications are incorporated by reference herein in their entirety. Suitable nasal pump spray devices include the VP50, VP70 and VP100 models available from Valois S.A. in Marly Le Roi, France and the 50, 70 and 100 μl nasal pump sprays available from Pfeiffer GmbH in Radolfzell, Germany, although other models and sizes can be employed. In the aforementioned embodiments, a pharmaceutical dose or dose unit in accordance with the invention can be present within the metering chamber of the metering pump or valve.

The compositions can be formulated as a suppository, with traditional binders and carriers such as triglycerides, microcrystalline cellulose, gum tragacanth or gelatin. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in: Remington's Pharmaceutical Sciences” by E.W. Martin, the contents of which are hereby incorporated by reference herein. Such compositions will contain a therapeutically effective amount of a peptide of the invention, preferably in a substantially purified form, together with a suitable amount of carrier so as to provide the form for proper administration to the subject.

Microglial modulators of the invention, polynucleotides encoding them, and compositions comprising them, can be delivered to a cell either through direct contact with the cell or via a carrier means. Carrier means for delivering microglial modulators and compositions to cells are known in the art and include, for example, encapsulating the composition in a liposome moiety. Another means for delivery comprises attaching the microglial modulator to a protein or nucleic acid that is targeted for delivery to the target cell. U.S. Pat. No. 6,960,648 and Published U.S. Patent Application Nos. 20030032594 and 20020120100 disclose amino acid sequences that can be coupled to another composition and that allows the composition to be translocated across biological membranes.

The route of administration of the pharmaceutical composition will depend on the disease or condition to be treated. Suitable routes of administration include, but are not limited to, parenteral injections, e.g., intradermal, intravenous, intramuscular, intralesional, subcutaneous, intrathecal, and any other mode of injection as known in the art. Although the bioavailability of peptides administered by other routes can be lower than when administered via parenteral injection, by using appropriate formulations it is envisaged that it will be possible to administer the compositions of the invention via transdermal, oral, rectal, vaginal, topical, nasal, inhalation and ocular modes of treatment. In addition, it may be desirable to introduce the pharmaceutical compositions of the invention by any suitable route, including intraventricular and intrathecal injection; intraventricular injection may be facilitated by an intraventricular catheter, for example, attached to a reservoir. Pulmonary administration can also be employed, e.g., by use of an inhaler or nebulizer.

In some embodiments, the route of administration is improved by encapsulating the pharmaceutical agent in nanoparticles, such as to protect the encapsulated drug from biological and/or chemical degradation, and/or to facilitate transport to the brain thereby targeting microglia.

In one embodiment, compositions of the present invention comprise compounds used for attenuating depression condition or disease in a subject in need thereof. In some embodiments, composition of the present invention is used in combination with electroconvulsive therapy.

In some embodiments, compositions for use in the methods of this invention comprise solutions or emulsions, which in some embodiments are aqueous solutions or emulsions comprising a safe and effective amount of the compounds of the present invention and optionally, other compounds, intended for topical intranasal administration. In some embodiments, the compositions comprise from about 0.01% to about 10.0% w/v of a subject compound, more preferably from about 0.1% to about 2.0, which is used for systemic delivery of the compounds by the intranasal route.

In another embodiment, the pharmaceutical compositions are administered by intravenous, intra-arterial, or intramuscular injection of a liquid preparation. In some embodiments, liquid formulations include solutions, suspensions, dispersions, emulsions, oils and the like. In one embodiment, the pharmaceutical compositions are administered intravenously, and are thus formulated in a form suitable for intravenous administration. In another embodiment, the pharmaceutical compositions are administered intra-arterially, and are thus formulated in a form suitable for intra-arterial administration. In another embodiment, the pharmaceutical compositions are administered intramuscularly, and are thus formulated in a form suitable for intramuscular administration.

Further, in another embodiment, the pharmaceutical compositions are administered topically to body surfaces, and are thus formulated in a form suitable for topical administration. Suitable topical formulations include gels, ointments, creams, lotions, drops and the like. For topical administration, the compounds of the present invention are combined with an additional appropriate therapeutic agent or agents, prepared and applied as solutions, suspensions, or emulsions in a physiologically acceptable diluent with or without a pharmaceutical carrier.

In one embodiment, pharmaceutical compositions of the present invention are manufactured by processes well known in the art, e.g., by means of conventional mixing, dissolving, granulating, dragee-making, levigating, emulsifying, encapsulating, entrapping or lyophilizing processes.

In one embodiment, pharmaceutical compositions for use in accordance with the present invention is formulated in conventional manner using one or more physiologically acceptable carriers comprising excipients and auxiliaries, which facilitate processing of the active ingredients into preparations which, can be used pharmaceutically. In one embodiment, formulation is dependent upon the route of administration chosen.

In one embodiment, injectables of the invention are formulated in aqueous solutions. In one embodiment, injectables, of the invention are formulated in physiologically compatible buffers such as Hank's solution, Ringer's solution, or physiological salt buffer. In some embodiments, for transmucosal administration, penetrants appropriate to the barrier to be permeated are used in the formulation. Such penetrants are generally known in the art.

In one embodiment, the preparations described herein are formulated for parenteral administration, e.g., by bolus injection or continuous infusion. In some embodiments, formulations for injection are presented in unit dosage form, e.g., in ampoules or in multi-dose containers with optionally, an added preservative. In some embodiments, compositions are suspensions, solutions or emulsions in oily or aqueous vehicles, and contain formulatory agents such as suspending, stabilizing and/or dispersing agents.

The compositions also comprise, in some embodiments, preservatives, such as benzalkonium chloride and thimerosal and the like; chelating agents, such as edetate sodium and others; buffers such as phosphate, citrate and acetate; tonicity agents such as sodium chloride, potassium chloride, glycerin, mannitol and others; antioxidants such as ascorbic acid, acetylcysteine, sodium metabisulfite and others; aromatic agents; viscosity adjustors, such as polymers, including cellulose and derivatives thereof; and polyvinyl alcohol and acid and bases to adjust the pH of these aqueous compositions as needed. The compositions also comprise, in some embodiments, local anesthetics or other actives. The compositions can be used as sprays, mists, drops, and the like.

In some embodiments, pharmaceutical compositions for parenteral administration include aqueous solutions of the active preparation in water-soluble form. Additionally, suspensions of the active ingredients, in some embodiments, are prepared as appropriate oily or water-based injection suspensions. Suitable lipophilic solvents or vehicles include, in some embodiments, fatty oils such as sesame oil, or synthetic fatty acid esters such as ethyl oleate, triglycerides or liposomes. Aqueous injection suspensions contain, in some embodiments, substances, which increase the viscosity of the suspension, such as sodium carboxymethyl cellulose, sorbitol or dextran. In another embodiment, the suspension also contains suitable stabilizers or agents which increase the solubility of the active ingredients to allow for the preparation of highly concentrated solutions.

In another embodiment, the active compound can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid).

In another embodiment, the pharmaceutical composition delivered in a controlled release system is formulated for intravenous infusion, implantable osmotic pump, transdermal patch, liposomes, or other modes of administration. In one embodiment, a pump is used (see Langer, supra; Sefton, CRC Crit. Ref. Biomed. Eng. 14:201 (1987); Buchwald et al., (1980); Saudek et al., (1989). In another embodiment, polymeric materials can be used. In yet another embodiment, a controlled release system can be placed in proximity to the therapeutic target, thus requiring only a fraction of the systemic dose (see, e.g., Goodson, in Medical Applications of Controlled Release, supra, vol. 2, pp. 115-138 (1984). Other controlled release systems are discussed in the review by Langer (1990).

In some embodiments, the active ingredient is in powder form for constitution with a suitable vehicle, e.g., sterile, pyrogen-free water-based solution, before use. Compositions are formulated, in some embodiments, for atomization and inhalation administration. In another embodiment, compositions are contained in a container with attached atomizing means.

In one embodiment, the preparation of the present invention is formulated in rectal compositions such as suppositories or retention enemas, using, e.g., conventional suppository bases such as cocoa butter or other glycerides.

In some embodiments, pharmaceutical compositions suitable for use in context of the present invention include compositions wherein the active ingredients are contained in an amount effective to achieve the intended purpose. In some embodiments, a therapeutically effective amount means an amount of active ingredients effective to prevent, alleviate or ameliorate symptoms of disease or prolong the survival of the subject being treated.

In one embodiment, determination of a therapeutically effective amount is well within the capability of those skilled in the art.

In some embodiments, preparation of effective amount or dose can be estimated initially from in vitro assays. In one embodiment, a dose can be formulated in animal models and such information can be used to more accurately determine useful doses in humans.

In one embodiment, toxicity and therapeutic efficacy of the active ingredients described herein can be determined by standard pharmaceutical procedures in vitro, in cell cultures or experimental animals. In one embodiment, the data obtained from these in vitro and cell culture assays and animal studies can be used in formulating a range of dosage for use in human. In one embodiment, the dosages vary depending upon the dosage form employed and the route of administration utilized. In one embodiment, the exact formulation, route of administration and dosage can be chosen by the individual physician in view of the patient's condition. [See e.g., Fingl, et al., (1975)].

In one embodiment, dosing can be of a single or a plurality of administrations, with course of treatment lasting from several days to several weeks or until cure is affected or diminution of the disease state is achieved. In another embodiment, said dosing can depend on severity and responsiveness of the condition to be treated.

In one embodiment, the amount of a composition to be administered will, of course, be dependent on the subject being treated, the severity of the affliction, the manner of administration, the judgment of the prescribing physician, etc.

In one embodiment, compositions including the preparation of the present invention formulated in a compatible pharmaceutical carrier are also be prepared, placed in an appropriate container, and labeled for treatment of an indicated condition.

In some embodiment, the term “therapeutically effective amount” refers to a concentration of a microglia modulator selected from the group consisting of: compounds blocking LAG-3, Cd180, TDO2, B7-2, PD-L1, PLA2E4 or any combination thereof, effective to treat a disease or disorder in a mammal. The term “a therapeutically effective amount” refers to an amount effective, at dosages and for periods of time necessary, to achieve the desired therapeutic result. The exact dosage form and regimen would be determined by the physician according to the patient's condition.

As used herein, the terms “subject” or “individual” or “animal” or “patient” or “mammal,” refers to any subject, particularly a mammalian subject, for whom therapy is desired, for example, a human.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a,” “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In the description and claims of the present application, each of the verbs, “comprise,” “include” and “have” and conjugates thereof, are used to indicate that the object or objects of the verb are not necessarily a complete listing of components, elements or parts of the subject or subjects of the verb.

Other terms as used herein are meant to be defined by their well-known meanings in the art.

Unless specifically stated or obvious from context, as used herein, the term “or” is understood to be inclusive.

It is to be noted that the term “a” or “an” entity, refers to one or more of that entity; for example, “a polypeptide,” is understood to represent one or more polypeptides. As such, the terms “a” (or “an”), “one or more,” and “at least one” can be used interchangeably herein.

Throughout this specification and claims, the word “comprise,” or variations such as “comprises” or “comprising,” indicate the inclusion of any recited integer or group of integers but not the exclusion of any other integer or group of integers.

As used herein, the term “consists essentially of,” or variations such as “consist essentially of” or “consisting essentially of,” as used throughout the specification and claims, indicate the inclusion of any recited integer or group of integers, and the optional inclusion of any recited integer or group of integers that do not materially change the basic or novel properties of the specified method, structure or composition.

As used herein, the terms “comprises,” “comprising,” “containing,” “having” and the like can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments. In one embodiment, the terms “comprises,” “comprising, “having” are/is interchangeable with “consisting”.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Generally, the nomenclature used herein, and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds.) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Strategies for Protein Purification and Characterization—A Laboratory Course Manual” CSHL Press (1996); all of which are incorporated by reference. Other general references are provided throughout this document.

### Materials and Methods

### Subjects

Subjects were 6-7 months old male WT C57BL/6 mice. Animals were housed in air-conditioned rooms (23° C.), with food and water ad libitum, and were kept in a reversed light/dark cycle, with lights off from 9 a.m. to 9 p.m. All experiments were approved by the Hebrew University of Jerusalem Ethics Committee on Animal Care, and use.

### Experimental Design

Microglia Manipulations with CSF-1 Antagonist or Minocycline Administration

To induce microglia depletion, subjects were divided into two groups, treated with either a diet containing 1,200 mg/g PLX5562 (Plexxikon Inc., U.S.A.), a selective CSF1 receptor kinase inhibitor, which when given chronically (i.e., for more than 2-3 weeks) induces near-complete microglial depletion (Danger et al., 2015), or with a control diet (identical diet excluding PLX5562). To specifically block the activation of microglia, mice were treated with minocycline (Sigma, Israel), administrated via the drinking water at a dose of 40 mg/kg/day. This dose regimen has been previously found to be effective in counteracting chronic stress-induced microglial alterations and behavioral changes (Hinwood M, Tynan R J, Charnley J L, Beynon S B, Day T A, Walker F R. Chronic stress induced remodeling of the prefrontal cortex: structural re-organization of microglia and the inhibitory effect of minocycline. Cerebral Cortex, 23: 1784-1797, 2012).

### Chronic Unpredictable Stress (CUS) Procedure

The CUS schedule comprised daily exposure to two stressors in a random order over a 5-week period. The list of CUS stressors included: cage shaking for 1 h with loud music and lights on, lights on during the entire night (12 h), lights-off for 3 h during the daylight phase, flashing (stroboscopic) light for 6 h, placement in 4° C. cold room for 1 h, mild restraint (in small ventilated cages) for 2 h, 45° cage tilt for 14 h, wet sawdust in the cage for 14 h, exposure to rat odor for 2 h, noise in the room for 3 h, and water deprivation for 12 h during the dark period. Another group of subjects administered with the control diet did not undergo the CUS procedure and served as an untreated (non-stressed) control group.

### ECT Procedure

ECT was applied following CUS exposure and verification of the development of depressive-like symptoms. ECT was applied 3-times per week for 2.5 weeks. Before each treatment, mice were lightly anesthetized with isoflurane, and administered a single shock via ear clip electrodes, using a Ugo Basile ECT Unit (Varese, Italy). The following shock parameters were used: frequency=100 pulses per sec, current=18 mA, shock duration=0.3 sec, and pulse width=0.5 ms. To control for the effects of ECT, half of the mice in each experiment underwent SHAM treatment, in which they were exposed to the same procedure, but no current was applied through the electrodes.

### Behavioral Measurements

### Sucrose Preference Test

Following baseline adaptation to sucrose for 3-4 days, mice were presented in the beginning of the dark circadian phase with two graduated drinking tubes, one containing tap water and the other 1% sucrose solution for 4 h. Sucrose preference was calculated as the percentage of sucrose consumption out of the total drinking volume.

### Social Exploration

Each subject was placed in an observation cage and allowed to habituate to the cage for 15 min, following which a male juvenile was placed in the cage. Social exploration, defined as the time of near contact between the nose of the subject and the juvenile conspecific, was then recorded for 2 min, using computerized in-house software.

### The Porsolt Forced Swim Test

Mice were placed in a plastic cylinder (with a height of 30 cm and diameter of 20 cm), filled with 25° C. water. The time spent immobile, defined as the absence of all movement except for motions required to maintain head above water were recorded for 6 min, and automatically analyzed off-line using the EthoVision software (Noldus).

### Immunohistochemistry

Animals were perfused transcardially with cold phosphate-buffered saline (PBS) followed by 4% paraformaldehyde in 0.1 M PBS, and the brains were quickly removed and placed in 4% paraformaldehyde. After 24 h, the brains were placed in 30% sucrose solution in PBS for 48 h and then frozen in OCT. Coronal sections (8 μm, 14 μm, or 50 μm) were serially cut along the rostro-caudal axis of the dorsal hippocampus, using a cryostat (Leica, Wetzlar, Germany), and mounted on slides.

Microglia were visualized using a primary antibody to the microglial marker ionized calcium-binding adapter molecule-1 (Iba-1) (rabbit anti Iba-1 1:250, Wako, Japan), followed by a secondary antibody (goat anti rabbit, 1:200; Invitrogen, Carlsbad, Calif., USA). The rate of neurogenesis in the hippocampus was measured by staining for doublecortin (DCX), using guinea pig anti-DCX (1:1,000, Millipore, Chemicon, Tamecula, Calif., USA) as the primary antibody, and biotin-SP-conjugated donkey anti-Guinea pig (1:200; Jackson Laboratories, West grove, PA, USA) as the secondary antibody, with final visualization using a conjugated streptavidin Ab (Jackson Laboratories, West grove, PA, USA). Rabbit-anti P2yr12 1:250 was also used to visualize microglia (AnaSpec, Fremont, Calif., USA), followed by a secondary antibody (goat anti rabbit, 1:200; Invitrogen, Carlsbad, Calif., USA). Microglial LAG-3 was visualized using the monoclonal LAG-3 MABF954 clone 4-10-C9 antibody, 1:200 (Millipore, Mass., USA).

### Image Analysis

Images were captured using a Nikon Eclipse microscope and camera. Cells were manually counted using 10× magnification in a defined area exclusively containing the dentate gyrus (DG) or CA3 region of the dorsal hippocampus for each slide, using Nikon Imaging Elements Software (NIS-Elements). Four sections of each brain were counted. Confocal images were captured using an Olympus FV-1000 confocal microscope. Slices were imaged at 0.165-0.2 μm/pixel in the XY dimension and at 0.5 μm steps in the Z dimension, using collapsed z-stacks. Microglia cell processes length was measured by capturing images at 40× magnification and by manual tracing of the processes of all Iba-1+ cells in these sections, using the Image)/FIJI software. Microglia contacts with DCX-stained cells were quantified using z-stacks compiled by Image)/FIJI software, and observation of spatial overlap between fluorescent labels defined contact regions. Contacts were recorded if spatial overlap was observed on the body or on the dendrite branches of P2YR12-positive cells. The quantity of contacts per cell were recorded in each hippocampus regional slide's images.

### Real-Time Quantitative PCR

Mice were sacrificed by decapitation. Each brain was quickly removed on an ice-cold glass plate, and the hippocampus was dissected out under a binocular. Tissues were weighed, flash frozen in liquid nitrogen, and stored in −80° C. until RNA extraction. RNA was extracted using PerfectPure RNA extraction kit (5 PRIME, Darmstadt, Germany). RNA samples (2 μg) were reverse transcribed using the QuantiTect Reverse Transcription Kit from Qiagen (Hilden, Germany), including DNase treatment of contaminating genomic DNA. Expression of mRNA was determined by qPCR, using glyceraldehyde-3-phosphate dehydrogenase (Gapdh) as a normalizing gene. The following list of gene transcripts was validated: Ionized calcium-binding adapter molecule 1 (Iba1), the Purinergic receptor P2yr12, Lymphocyte-activation gene 3 (Lag-3), Cd180, tryptophan 2,3-dioxygenase (Tdo2), Phospholipase A2 Group IVE (Plag24e) Sox11, and Dopamine Receptor D1 (Drd1). Primers were designed using PrimerQuest IDT (Integrated DNA Technologies, Inc, San Diego, Calif., USA). The following primers were used, Gapdh, Forward: TCTCCCTCACAATTTCC (SEQ ID NO: 1); Reverse: GGGTGCAGCGAACTTTA (SEQ ID NO: 2). Drd1, Forward: CTTCTGGAAGATGGCTCCTAAC (SEQ ID NO: 3); Reverse: CCCTAAGAGAGTGGACAGGATA (SEQ ID NO: 4). Tdo2, Forward: CATCGTGTGGTGGTCATCTT (SEQ ID NO: 5); Reverse: CTGATGCTGGAGACAGGTATTC (SEQ ID NO: 6). Iba1, Forward: GACGTTCAGCTACTCTGACTTT (SEQ ID NO: 7); Reverse: GTTGGCCTCTTGTGTTCTTTG (SEQ ID NO: 8). Lag-3, Forward: TCATCACAGTGACTCCCAAATC (SEQ ID NO: 9); Reverse: GCCACACAAATCTTTCCTTTCC (SEQ ID NO: 10). Cd180, Forward: CTCCGAAACCTGTCTCACTTAC (SEQ ID NO: 11); Reverse: GTTCTAGCTGAGGGCATTCTT (SEQ ID NO: 12). Sox11, Forward: CTCCATCACTCGGCTTTCTTAT (SEQ ID NO: 13); Reverse: CTCTCTTCCAAGTGTCCACAAA (SEQ ID NO: 14). Plag24e, Forward: CAGGAACCCATACTGTGAAGA (SEQ ID NO: 15); Reverse: GCTGGTAGGAGAGTGTGATAAAT (SEQ ID NO: 16), and P2yr12 Forward: CCTTAACACTAGAGGCAGCAA (SEQ ID NO: 17); Reverse: CATTCAAGCAGCAGGCATTT (SEQ ID NO: 18). Normal and mock reversed transcribed samples (in the absence of reverse transcriptase), as well as no template controls (total mix without cDNA) were run for each of the examined mRNAs. qPCR reactions were subjected to an initial step of 15 min at 95° C. to activate the HotStar Taq DNA polymerase, followed by 40 cycles consisting of 15 s at 94° C., 30 s at 60° C. and 30 s at 72° C. Fluorescence was measured at the end of each elongation step. Data were collected and analyzed using the StepOnePlus instrument and software (Thermo Fischer Scientific), and a threshold cycle value Ct was calculated from the exponential phase of each PCR sample. Expression levels of mRNAs were calculated and expressed in relative units of SYBR Green fluorescence.

### RNA Sequencing

RNA Sequencing, PolyA based mRNA was selected using oligodT beads, followed by fragmentation, first strand and second strand synthesis reactions. Illumina libraries were constructed while performing the end repair, A base addition, adapter ligation and PCR amplification steps with SPRI beads cleanup in between steps. Indexed samples were pooled and sequenced in an Illumina HiSeq 2500 machine in a single read mode.

Bioinformatics analysis. Adapters were trimmed using the cutadapt tool. Following adapter removal, reads that were shorter than 40 nucleotides were discarded (cutadapt option −m 40). Reads that had either a percentage of Adenine bases above 50% or a percentage of Thymine bases above 50% were discarded using a custom script. TopHat (v2.0.10) was used to align the reads to the mouse genome (mm10). Counting reads on mm10 refseq genes (downloaded from igenomes) was done with HTSeq-count (version 0.6.1p1). Differential expression analysis was performed using DESeq2 (1.6.3). Raw p values were adjusted for multiple testing (q-value, false discovery rate).

### Statistical Analysis

All data are presented as mean±SEM. Statistical comparisons were computed using SPSS 19.0 software and consisted of t-tests, one-way and two-way analyses of variance (ANOVAs) (using Wilks' Lambda), followed by the Fisher's least significant difference (LSD) post hoc analyses, or Bonferroni-corrected, or t-tests, when appropriate.

### Example 1

### ECT Increases the Number and Size of Microglia in CUS-Exposed Mice

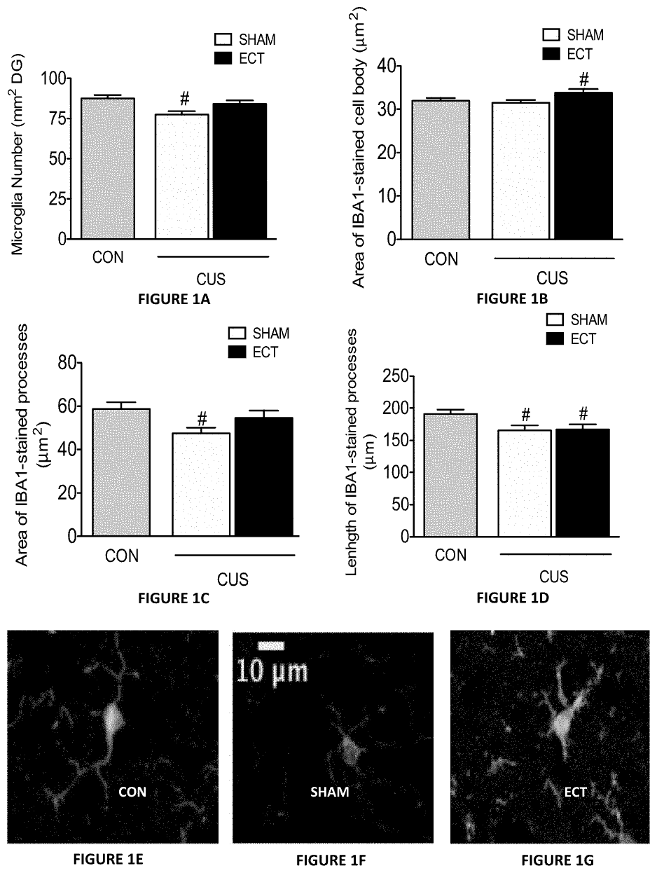

The inventors first assessed the effects of ECT on microglial morphological activation status following exposure to chronic unpredictable stress (CUS)—according to an established model in mice. While previous studies showed that ECT affects the morphology of microglia cells in normal mice (Jansson et al., 2009), the effects of ECT on microglial morphology in chronically stressed, “depressed-like” mice were not shown. The inventors analyzed the morphometric changes in hippocampal dentate gyrus (DG) microglia of mice exposed to five weeks of CUS followed by 2.5 weeks of ECT or SHAM treatment (in the latter, mice were anesthetized and connected to the stimulating electrodes, but no current was passed). The inventors' analysis revealed significant CUS-induced reductions in the number of microglia in the DG of SHAM-treated mice, as compared to non-stressed controls. This reduction was reversed by ECT (FIG. 1A). ECT-treated mice showed significantly enlarged IBA1-stained cell bodies of microglia in the DG of the hippocampus (which is a characteristic of microglial activation) (FIG. 1B). ECT also reversed the reduction in overall microglial cell area that was seen in CUS-exposed SHAM-treated mice (FIG. 1C). Finally, the length of microglial IBA-1-immunostained processes in the DG of CUS-exposed mice was significantly reduced in both the SHAM and the ECT groups (FIG. 1D).

### Example 2

### Depletion of Brain Microglia Blocks the Anti-Depressive Effects of ECT

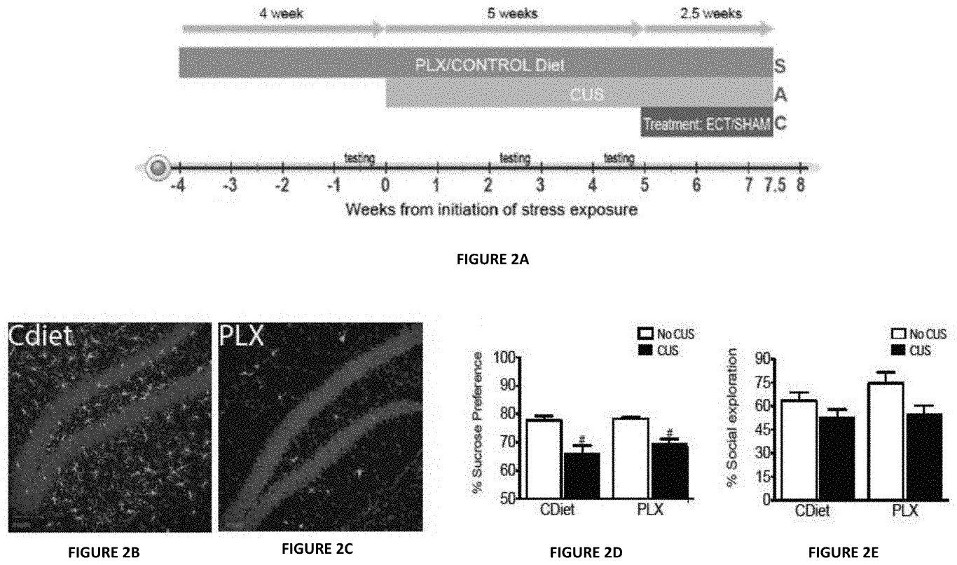

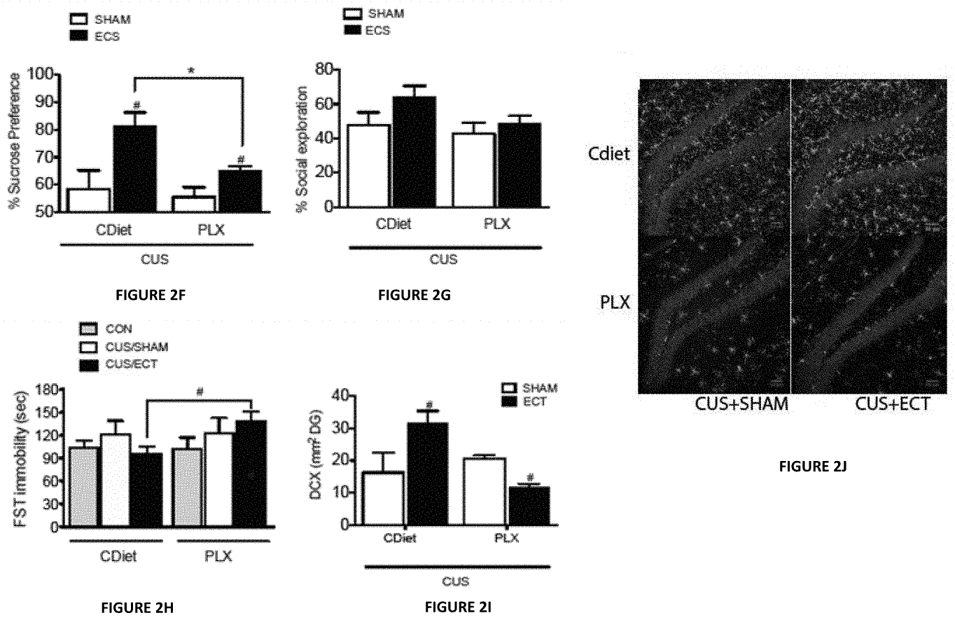

To examine whether the effects of ECT on microglia are relevant to the anti-depressive effect of this procedure, the inventors examined the effects of ECT in mice with near-complete microglia depletion (FIG. 2A). Depletion was induced by a three weeks exposure to a diet containing PLX5622—an antagonist of the receptor for CSF-1 (which is essential for microglial survival). Control animals received the same diet without PLX5622 (CDiet). This procedure resulted in near-complete depletion of all brain microglia, including microglia in the hippocampus (FIGS. 2B-2C). The microglia depletion did not cause any depressive-like symptoms by itself. Specifically, it did not reduce sucrose preference (a measure of anhedonia) (FIG. 2D) or social exploration/activity (another common depressive symptom) (FIG. 2E) or the immobility in the forced swim stress (a measure of despair) (FIG. 2H—two left columns). The microglia depletion also did not influence the development of CUS-induced depression (FIGS. 2D-2E), suggesting that in the complete absence of microglia other cells can promote the development of depression. However, the finding that mice treated with either control diet or PLX5622 developed similar depressive-like behaviors allowed the inventors to investigate the role of microglia in the anti-depressive effect of ECT. Specifically, the inventors examined whether depressed-like (CUS-exposed) mice with microglia-depletion would exhibit an anti-depressive effect of a 2.5-week course of ECT. The inventors found that microglial depletion markedly attenuated the anti-depressive effect of ECT. Specifically, although ECT significantly increased sucrose preference in both groups, this increase was significantly greater in the CDiet group (in which sucrose preference was elevated to the normal levels that are usually observed in non-stressed mice) than in the PLX-treated group (FIG. 2F). A similar effect was shown in the social exploration test (FIG. 2G). In the Porsolt forced swim test, the effect of ECT (in reducing immobility time, i.e., despair-like behavior) was also significantly greater in the CDiet than in the PLX-treated group (FIG. 2H). Furthermore, the inventors found that microglia depletion also completely prevented the beneficial effect of ECT on hippocampal neurogenesis (FIGS. 2I-2J), which is considered a major target for all antidepressant drugs and procedures.

### Example 3

### ECT Anti-Depressive Effect Involves Regulation of Inhibitory Immune Checkpoints Transcription

To elucidate the potential mechanisms underlying the anti-depressive effect of ECT in CDiet mice and its attenuation in the PLX-treated mice, the inventors explored the group differences in transcriptional regulation in the hippocampus, which is known to be involved in regulation of emotional and cognitive processes, as well as in mediating the therapeutic effects of ECT. RNA sequencing analysis revealed that in the CUS-exposed (depressed-like) CDiet groups, a total of 15 hippocampal transcripts were modulated by ECT. These genes were significantly differentially regulated between the SHAM vs. ECT mice, with 8 genes showing down-regulation, and 7 showing up-regulation (q<0.32, with a cutoff of ±1.3-fold change; table 1). Remarkably, in the PLX-treated groups no genes were differentially regulated between CUS-exposed SHAM vs. ECT mice, demonstrating that the effects of ECT on gene transcription occur only in the presence of intact brain microglia. Importantly, 3 out of the 7 genes that showed ECT-induced significant down-regulation are known to be inhibitory immune checkpoints in the periphery, including Lag-3, Cd180, and Tdo2. A fourth gene, Pla2g4e encoding the enzyme PLA2G4E, was identified as the calcium-dependent acyltransferase that produces N-acyl-phosphatidylethanolamines (which are the precursors of N-acyl ethanolamines including N-palmitoylethanolamine and the endocannabinoid, N-arachidonoylethanolamine (anandamide). Evidence suggested that increased endocannabinoid levels are associated with immune and microglial suppression, thus, a decrease in Pla2g4e transcript (and protein) is associated with immune/microglial activation due to expected lower levels of endocannabinoids. These findings suggest that the anti-depressive effect of ECT involves the breaking of brain immune checkpoints.

The up-regulated genes were found to include: Sox11, which is critical for hippocampal neurogenesis, as well as dopamine receptor D1 (Drd1) and synaptic vesicle glycoprotein 2C (Sv2c), which mediates and facilitates neurotransmission in the dopaminergic system.

TABLE 1
Gene transcript significantly differentially expressed
in ECT vs. SHAM in control diet CUS-exposed mice

| | | | | |
|---|---|---|---|---|
| | | | | False Discovery |
| Gene Symbol | Gene Name | Log Ratio | p-value | Rate (q-value) |

Immune checkpoint

| | | | | |
|---|---|---|---|---|
| Lag-3 | lymphocyte activating gene 3 | −1.831 | 0.000745 | 0.319 |
| Cd-180 | Cd180 molecule | −1.613 | 0.000858 | 0.319 |
| Tdo2 | tryptophan 2,3-dioxygenase | −1.471 | 0.000123 | 0.137 |

Immune system-related

| | | | | |
|---|---|---|---|---|
| Csf2rb2 | Colony stimulating factor 2 | −1.834 | 2.3E−07 | 0.002 |
| | receptor beta common subunit | | | |
| H2-D1 | Major histocompatibility | −1.408 | 0.000785 | 0.319 |
| | complex, class I, A | | | |
| B2m | Beta-2 microglobulin | −1.554 | 0.000234 | 0.173 |

Endocannabinoid signaling

| | | | | |
|---|---|---|---|---|
| Pla2g4e | Phospholipase A2 group IVE | −1.554 | 0.00074 | 0.319 |

Transcription factors

| | | | | |
|---|---|---|---|---|
| Zcchc5 | Zinc finger CCHC-type | −1.725 | 5.9E−05 | 0.0987 |
| | containing 5 | | | |
| MafA | MafbZIP transcription factor A | −1.695 | 0.000203 | 0.173 |

Neurogenesis

| | | | | |
|---|---|---|---|---|
| Sox11 | SRY-box 11 | 1.434 | 5.5E−09 | 0.00011 |

Synaptic neurotransmission

| | | | | |
|---|---|---|---|---|
| Drd1 | Dopamine receptor D1 | 1.483 | 0.000232 | 0.173 |
| Sv2c | Synaptic vesicle glycoprotein 2C | 1.42 | 0.000166 | 0.157 |

Other

| | | | | |
|---|---|---|---|---|
| Noxred1 | NADP dependent oxidoreductase | 1.979 | 0.00093 | 0.319 |
| | domain containing 1 | | | |
| Serinc2 | Serine incorporator 2 | 1.54 | 0.000589 | 0.294 |
| Pdia4 | Protein disulfide isomerase | 1.442 | 0.000569 | 0.294 |
| | family A member 4 | | | |

The RNA sequencing analysis further revealed a major effect of the PLX treatment on hippocampal gene transcription, likely reflecting the consequences of microglial depletion. Specifically, a total of 390 genes were differentially regulated in CUS-exposed PLX- vs. CDiet SHAM-treated mice, of which 338 genes were down-regulated, and 52 were up-regulated (q<0.32, with a cutoff of ±1.3-fold change). Only two of the 15 genes whose transcription was reduced by ECT in the CDiet group were abolished by microglial depletion: Lag-3 and Cd-180, and are therefore possibly the only two microglia-enriched genes that were influenced by ECT. Given that the anti-depressive effects of ECT were completely dependent on the presence of microglia, changes in the transcription of these genes are the most likely mediators of ECT's anti-depressive and neurogenesis enhancing effects.

### Example 4

### ECT Anti-Depressive Effect Involves Breaking of Specific Brain Inhibitory Immune Checkpoints

TABLE 2
Regulation of immune/microglial checkpoint genes by ECT,
microglia depletion, and chronic stress
Table 2. Regulation of immune/microglial checkpoint genes
by ECT, microglia depletion, and chronic stress

| | | Log 2 | | | Corresponding | | |
|---|---|---|---|---|---|---|---|
| | | (fold change) | | | p-values | | |
| | | Con-Stress- | Con-Stress- | Con-Stress- | Con-Stress- | Con-Stress- | Con-Stress- |
| | | ECT vs. | Sham vs. | Sham vs. | ECT vs. | Sham vs. | Sham vs. |
| Gene | Enterase | Con-Stress- | PLX-Stress- | Con-No | Con-Stress- | PLX-Stress- | Con-No |
| transcript | gene name | Sham | Sham | Stress | Sham | Sham | Stress |
| Lag-3 | Lymphocyte | −0.872 | 1.968 | 0.617 | 0.001 | <0.0001 | 0.03 |
| | activation gene 3 | | | | | | |
| Cd180 | Cluster of | −0.690 | 1.892 | 0.475 | 0.001 | <0.0001 | 0.034 |
| | differentiation | | | | | | |
| | 180 | | | | | | |
| Tdo2 | Tryptophan 2,3- | −0.556 | −0.248 | −0.056 | <0.0001 | 0.086 | 0.717 |
| | dioxygenase | | | | | | |
| Cd86 | Cluster of | −0.493 | 2.092 | 0.301 | 0.041 | <0.0001 | 0.249 |
| | differentiation | | | | | | |
| | 86 (B7-2) | | | | | | |
| PD-L1 | Programmed | −0.306 | 0.164 | 0.422 | 0.066 | 0.338 | 0.022 |
| | cell death-ligand | | | | | | |
| | 1 | | | | | | |
| Pdcd1 | Programmed | −0.977 | −0.669 | 0.797 | 0296 | 0.417 | 0.428 |
| | cell death | | | | | | |
| | protein 1 | | | | | | |
| Ctla4 | Cytotoxic T- | −0.504 | 0.100 | 0.389 | 0.821 | 0.932 | 0.76 |
| | lymphocyte | | | | | | |
| | associated | | | | | | |
| | protein 4 | | | | | | |
| Havcr2 | T cell | −0.177 | 2.101 | 0.1 | 0.275 | <0.0001 | 0.57 |
| | immunoglobulin | | | | | | |
| | and mucin | | | | | | |
| | domain | | | | | | |
| | containing 3 | | | | | | |
| | (TIM-3) | | | | | | |
| Pdcd1lg2 | Programmed | 0.464 | −0.373 | 0.492 | 0.558 | 0.651 | 0.611 |
| | cell death 1 | | | | | | |
| | ligand 2 | | | | | | |
| Cd276 | Cluster of | .0123 | 0.019 | 0.102 | 0.252 | 0.865 | 0.389 |
| | differentiation | | | | | | |
| | 276 | | | | | | |
| Vtcn1 | V-set domain | 1.747 | −0.905 | −1.204 | 0.384 | 0.676 | 0.585 |
| | containing t-cell | | | | | | |
| | activation | | | | | | |
| | inhibitor | | | | | | |
| Cd80 | Cluster of | 0.672 | 0.371 | −0.505 | 0.28 | 0.596 | 0.46 |
| | differentiation | | | | | | |
| | 80 | | | | | | |
| Cd40 | Cluster of | −0.300 | 0.159 | 0.873 | 0.953 | 0.785 | 0.191 |
| | differentiation | | | | | | |
| | 40 | | | | | | |
| Lgals3 | Galactose | −0.18 | 0.011 | 0.171 | 0.599 | 0.975 | 0.65 |
| | specific lectin 3 | | | | | | |
| Tigit | T-cell | −0.176 | −0.804 | 0.024 | 0.737 | 0.105 | 0.966 |
| | immunoreceptor | | | | | | |
| | with ig and itims | | | | | | |
| | domains | | | | | | |

Given the finding that a substantial proportion of the gene transcripts that were differentially regulated by ECT are known to be inhibitory immune checkpoints, the inventors further explored the data from the RNA-Seq analysis with respect to all major inhibitory immune checkpoints. The inventors noted the effects of ECT, as well as the effects of chronic stress (CUS) and microglial depletion by PLX on the transcription of these molecules. Comparison between ECT vs. SHAM treatment in the CUS-exposed groups given control diet (CDiet) (representing the net ECT effect) revealed that in addition to the 3 genes previously mentioned in Table 1 (Lag-3, Cd180 and Tdo2), the expression of Cd86 (B7-2) was also significantly reduced by ECT (p<0.05 used as the statistical significance threshold). The expression of the Pd-L1 gene showed a statistical trend for reduction (p<0.066). The expression of none of these genes was altered by ECT in CUS-exposed PLX5622-treated mice, verifying the critical role of microglia in mediating the effects of ECT. The net effect of CUS exposure (represented by the comparison between CUS-exposed SHAM-treated group on control diet (CDiet) vs. No stress group) revealed significant increases in three immune checkpoint transcripts —Lag-3, Cd180 and Pd-L1 by chronic stress (p<0.05), suggesting that the transcription of these gene is elevated in depressed individuals (Table 2). The effects of microglial depletion following PLX5622-containing diet were determined by comparing the PLX-SHAM vs. the CDiet-SHAM groups. Microglial depletion induced significant effects on the transcription of 4 immune checkpoint genes, including Lag-3, Cd180, Cd86 and Tim-3 (Table 2). It should be noted that the first three were also influenced by ECT in CDiet mice (i.e., they represent direct microglial inhibitory checkpoints), whereas the forth (Tim-3) was not influenced by either ECT or by CUS, suggesting that the involvement of immune checkpoints in depression is specific to particular immune/microglial checkpoints.

### Example 5

### qPCR Validation of Immune/Microglial Checkpoint Genes Showing ECT-Induced Transcriptional Regulation Changes in RNA-Seq Analysis

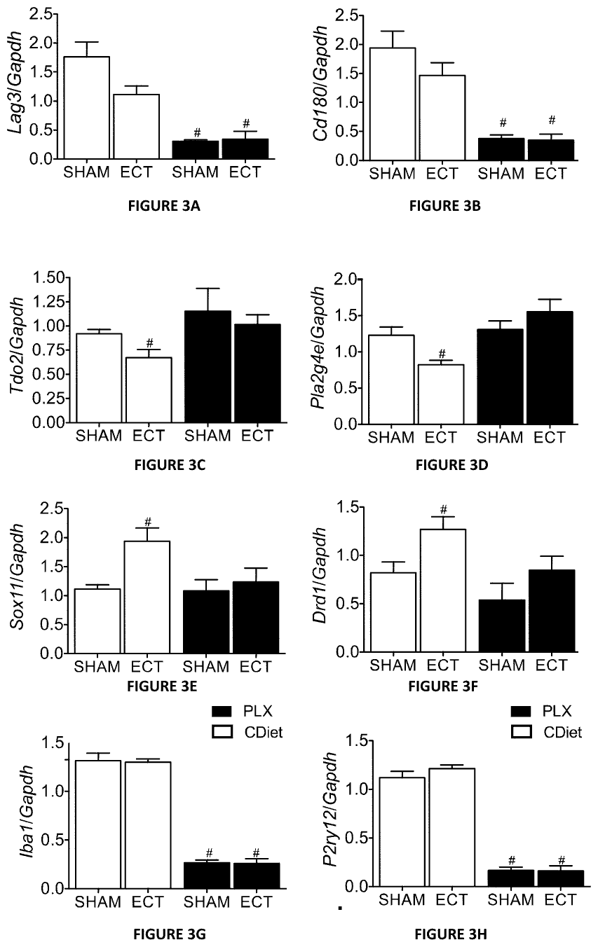

To validate the effects of ECT on immune/microglial checkpoint genes, as well as in genes known to be involved in neurogenesis and anti-depressive actions, the inventors analyzed the RNA expression of these molecules using qPCR methodology. Validation of the expression of the Lag-3 gene confirmed the significant transcription reduction following ECT in CDiet mice, whereas in PLX-treated mice Lag-3 transcription was almost completely abolished (suggesting that Lag-3 is a microglial-specific gene) (FIG. 3A). Similar findings were obtained with respect to Cd180 expression, but the finding of a reduction of this gene's expression following ECT did not reach statistical significance (FIG. 3B). qPCR validation of the Tdo2 (FIG. 3C) and pla2g4e expression confirmed that the expression of these genes was significantly reduced following ECT in the CDiet, but not in the PLX5622 groups. Furthermore, the expression of Tdo2 and pla2g4e was significantly increased in PLX5622-treated mice, indicating that these genes are mainly expressed by cells other than microglia, and that microglia depletion induces an increase in Tdo2 and pla2g4e gene expression. Validation of the expression of the Sox11 (FIG. 3E) and dopamine receptor D1 (Drd1) (FIG. 3F) genes (which are involved in neurogenesis and anti-depressive actions) confirmed that ECT increased the expression of both of these genes in SHAM-treated mice, but produced no effect in PLX5622-treated mice. The expression of either Sox11 or Drd1 was not affected by the microglia depletion procedure by itself (as expected from these neuronal markers). In contrast, validation of the expression of the Iba1 (FIG. 3G) and P2ry12 (FIG. 3H) genes confirmed that the expression of both of genes was significantly reduced by the PLX5622 procedure (as expected from previous findings that these are microglia-specific genes), but none of the two genes was affected by the ECT treatment.

### Example 6

### Blockade of Microglia Activation Attenuates ECT Effects on Behavior, Neurogenesis and Microglia-Newborn Neurons

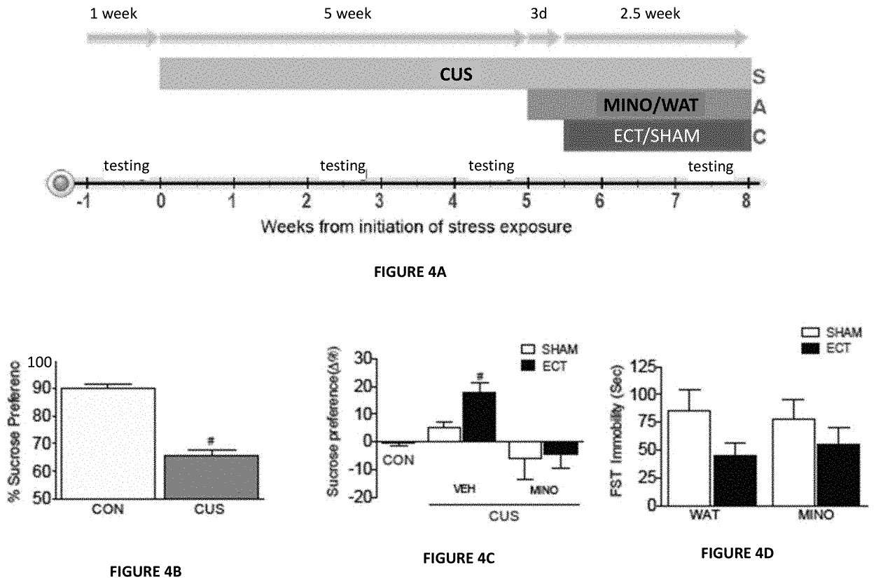

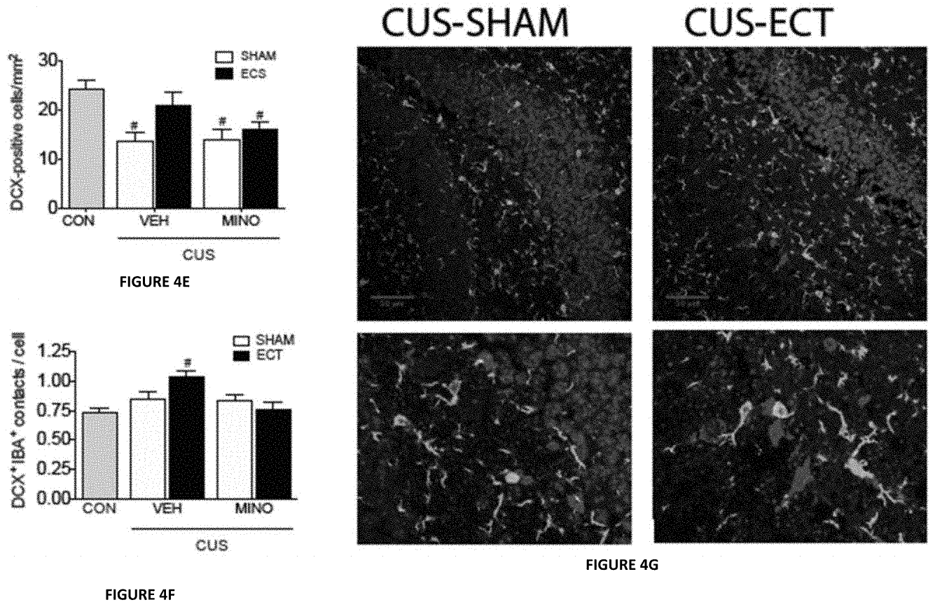

To determine whether microglia activation causally underlies the anti-depressant effects of ECT, as well as its effect on hippocampal neurogenesis, the inventors conducted an additional experiment in which the drug minocycline (MINO)—an established pharmacological blocker of microglia activation, was administered concomitantly with ECT (FIG. 4A). Five weeks following the initiation of CUS exposure (i.e., before the beginning of ECT treatment), chronically stressed mice showed a significant decrease in sucrose preference (FIG. 4B). In CUS-exposed mice, following 2.5 weeks of ECT, sucrose preference was significantly increased in the water-drinking but not the MINO-treated mice (FIG. 4C), however, the anti-depressive effect of ECT in the forced swim test was not influenced by MINO (FIG. 4D), suggesting that the inhibition of ECT-induced microglial activation by MINO partially prevented the therapeutic effect of ECT. To assess the effects of MINO on ECT-induced neurogenesis facilitation, the inventors analyzed the levels of adult neurogenesis in the DG by counting DCX-positive cells after treatment termination. As expected, CUS significantly reduced the levels of neurogenesis in the water-drinking SHAM-treated, and MINO-drinking SHAM-treated groups (p<0.001). However, the water-drinking ECT-treated group showed significantly increased neurogenesis levels (FIG. 4E), suggesting that ECT reversed the deleterious effect of CUS on neurogenesis. When ECT was applied together with MINO, the levels of neurogenesis remained decreased compared to non-stressed controls (FIG. 4E). To explore the possibility that alterations in microglial morphology were related to the effects on neurogenesis, the inventors analyzed the average number of microglial contacts with DCX-positive cells (including cell body and dendrites) in the DG. The inventors found that in the water-drinking group ECT significantly increased (p<0.001) the number of microglial contacts with neurogenic cells in the DG (FIG. 4F). No such increase was observed in MINO-treated mice, indicating that the facilitation of microglia-neurogenic cells interaction depends on microglia activation (FIGS. 4F-4G).

### Example 7

### LAG-3 Protein Expression by Microglia of the Hippocampal Dentate Gyrus

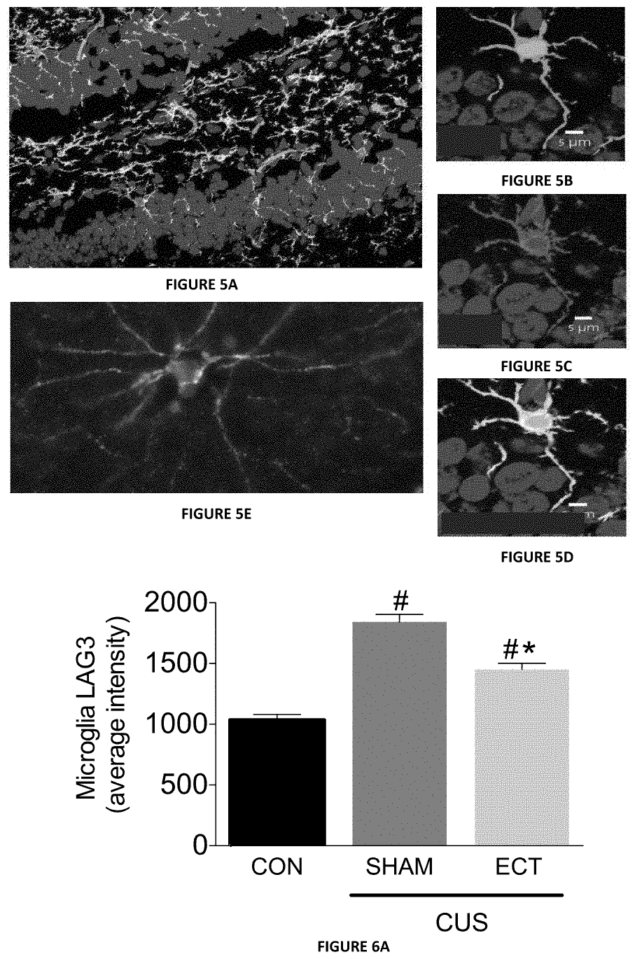

Assessment of the cellular localization of LAG-3 using a specific anti-LAG-3 antibody by immunohistochemical staining, revealed that LAG-3 expression is co-localized with a hallmark microglial marker (IBA-1), both in the murine (FIG. 5A-5D) and human (FIG. 5E) brain. It should be noted that all microglia were found to be positively labeled for LAG-3. Furthermore, LAG-3 expression was localized to the microglial cell membrane, both of the soma and the processes.

### Example 8

### ECT Normalizes the CUS-Induced Elevation of Microglial LAG-3 Protein

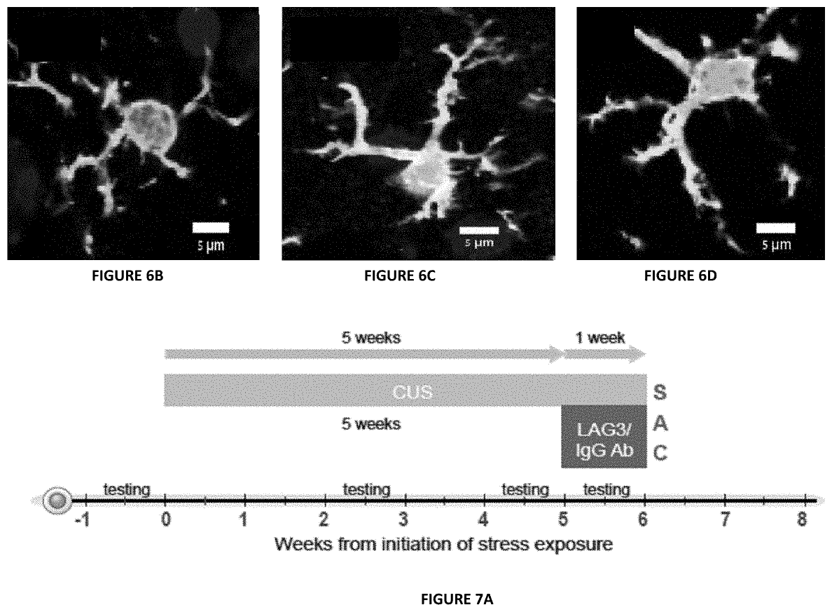

The intensity of LAG-3 staining was measured specifically within microglia cells (both soma and processes). In CUS-exposed (“depressed-like”) mice, receiving SHAM treatment the intensity of microglial LAG-3 was significantly elevated (FIGS. 6A-6B). This finding corroborated the finding of CUS-induced increase in Lag-3 transcription revealed by the RNA-Seq analysis (Table 2). ECT reduced the intensity of LAG-3 in microglia, in accordance with the effects of ECT on Lag-3 mRNA (Table 1 and FIG. 3A). Such a decrease in an inhibitory checkpoint molecule corroborated the inventors' finding that ECT induces morphological markers of microglial activation (FIG. 1).

### Example 9

### A Single LAG-3 Antibody Treatment Ameliorates CUS-Induced Depression

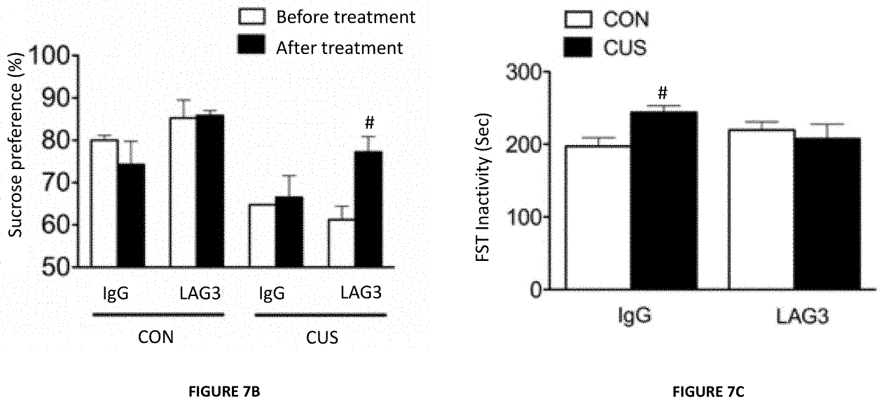

To provide direct evidence for the hypothesis that breaking the LAG-3 microglial checkpoint can serve as an anti-depressive treatment, the inventors assessed the effect of administrated anti-LAG-3 antibody, as compared with isotype IgG antibody, on CUS-induced anhedonia in the sucrose preference test. The anti-LAG-3 antibody (LEAF™ Purified anti-mouse CD223, Biolegend) was administered by means of intraperitoneal injection (i.p.) (100 μg) following 5 weeks of CUS exposure (FIG. 7A). Sucrose preference measured 3 days following the antibody administration, was significantly reduced in CUS-exposed IgG antibody-treated mice (FIG. 7B). However, treatment with the anti-LAG-3 monoclonal antibody (mAb) completely reversed the CUS-induced anhedonia (FIG. 7B). Despair in the Porsolt forced-swim test (reflected by the longer time spent in immobility) was significantly increased in CUS-exposed mice treated with IgG control (as compared with non-stressed IgG-treated mice), but this effect of CUS was also prevented by treatment with the anti-LAG-3 mAb (FIG. 7C). These findings demonstrated that anti-LAG-3 treatment can serve as a fast-acting anti-depressant procedure.

### Example 10

### Chronic LAG-3 Antibody Ameliorates CUS-Induced Depression More Efficiently than Escitalopram

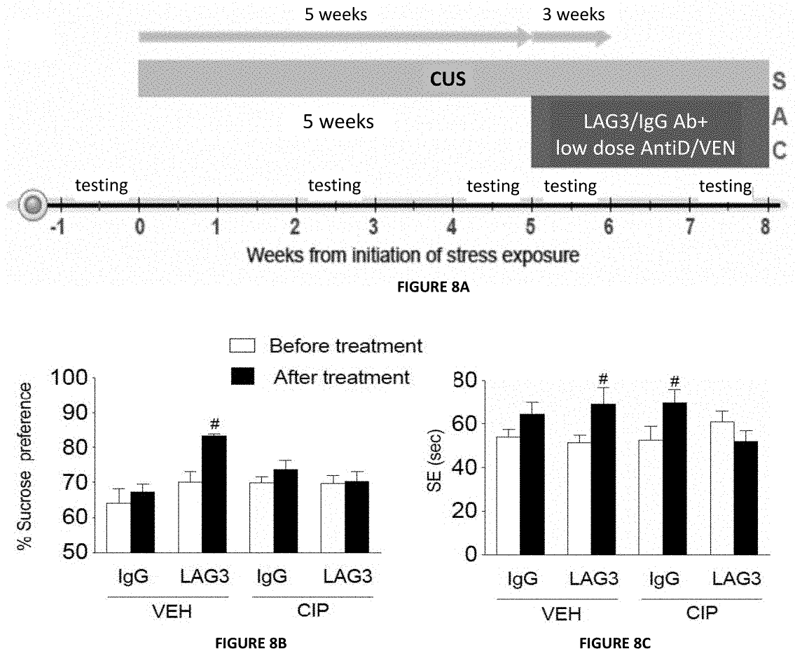

To provide further evidence for the anti-depressive effects of a chronic regimen of LAG-3 antibody administration, and to compare these effects with those of the SSRI drug escitalopram (Cipralex), the inventors conducted an additional experiment. The anti-LAG-3 antibody (LEAF™ Purified anti-mouse CD223, Biolegend, 100 μg) or isotype IgG antibody were administered by means of intraperitoneal injection (i.p.) following 5 weeks of CUS exposure (FIG. 1A). Injections were given every 4 days for a total of 6 injection (i.e., over a 3-weeks period, similarly to the regimen of ECT). Each of these groups was divided into two subgroups, injected daily (i.p.) with either Cipralex (CIP) or saline. Compared with the levels before treatment (i.e., in the “depressed-like” condition), sucrose preference was significantly elevated after treatment with the anti-LAG-3 antibody, whereas treatment with the IgG antibody or treatment with either escitalopram by itself (i.e., with the IgG antibody) or escitalopram together with the anti-LAG-3 antibody, had no such effect (FIG. 8B). These findings reflect the reversal of anhedonia by the anti-LAG-3 antibody, in a paradigm in which escitalopram produced no effect (probably because the 3 weeks period are not sufficient for effective treatment with this SSRI). Furthermore, the results demonstrate that the combination of anti-LAG-3 antibody with escitalopram mitigated the effect of the former, suggesting that this combination should be avoided in clinical practice. In the social exploration (SE) test, the levels of SE before treatment (i.e., in the “depressed-like” condition) were significantly elevated after treatment with the anti-LAG-3 antibody, as well as after escitalopram by itself (i.e., with the IgG antibody). In contrast, treatment with the IgG antibody or treatment with escitalopram together with the anti-LAG-3 antibody had no effect on SE (FIG. 8C). The findings on SE corroborate the anti-anhedonic effect of anti-LAG-3 antibody treatment, and demonstrate that escitalopram does have a beneficial effect on this measure. Moreover, the negative interaction between the anti-LAG-3 antibody and escitalopram is corroborated, emphasizing the conclusion that this combination should be avoided in clinical practice.

While the present invention has been particularly described, persons skilled in the art will appreciate that many variations and modifications can be made. Therefore, the invention is not to be construed as restricted to the particularly described embodiments, and the scope and concept of the invention will be more readily understood by reference to the claims, which follow.

1. A method for treating or attenuating a depressive disorder in a selective serotonin reuptake inhibitor (SSRI) non-treated subject, the method comprising administering to said subject a pharmaceutical composition comprising a therapeutically effective amount of at least one compound inhibiting a molecule selected from the group consisting of: lymphocyte-activation gene 3 (LAG-3), cluster of differentiation molecule 180 (CD180), tryptophan 2,3-dioxygenase (TDO2), cluster of differentiation molecule 86 (CD86/B7-2), programmed cell death ligand 1 (PD-L1), and Phospholipase A2 Group WE (PLA2G4E); and at least one pharmaceutically acceptable carrier or diluent; thereby treating or attenuating the depressive disorder in said subject.

2. The method of claim 1, further comprising the step of administering a second microglial activator to said subject.

3. The method of claim 2, wherein said second microglial activator is selected from the group consisting of: Macrophage colony-stimulating factor (M-CSF), Granulocyte macrophage colony-stimulating factor (GM-CSF), Interleukin 34 (IL-34), Granulocyte colony-stimulating factor (G-CSF), soluble LAG-3, and CX3C chemokine receptor 1 (CX3CR1) blockers.

4. The method of claim 1, further comprising selecting a subject having an increased level of at least one transcript or a protein product thereof compared to a baseline, wherein said transcript or a protein product thereof is selected from the group consisting of: LAG-3, CD180, TDO2, CD86/B7-2, PD-L1, and PLA2G4E.

5. The method of claim 4, wherein said transcript or a protein product thereof is detected in a sample of said subject, wherein said sample comprises: whole blood, peripheral blood mononuclear cells (PBMCs), isolated T cells, isolated dendritic cells, or isolated monocytes.

6. The method of claim 1, further comprising selecting a subject having a low inflammatory state, and optionally said low inflammatory state is reflected by plasma C-reactive protein (CRP) lower than 3 mg/L.

7. (canceled)

8. The method of claim 6, wherein said selecting a subject having a low inflammatory state is determining the plasma level of at least one inflammatory marker selected from CRP, IL-6 and TNFα, wherein a level of any one of: (i) less than 3 mg/L CRP, (ii) less than 2.0 pg/ml IL-6, (iii) less than 3.8 pg/ml TNFα, and (iv) combination thereof, indicates the subject has a low neuroinflammatory state suitable for treatment by said inhibitory-compound.

9. The method of claim 1, wherein said depressive disorder is selected from the group consisting of: unipolar major depressive episode, major depressive disorder, dysthymic disorder, treatment-resistant depression, bipolar depression, adjustment disorder with depressed mood, cyclothymic disorder, melancholic depression, psychotic depression, post-schizophrenic depression, depression due to a general medical condition, post-viral fatigue syndrome, and chronic fatigue syndrome.

10. The method of claim 1, wherein said at least one compound targets CD180 or PLA2G4E.

11. (canceled)

12. The method of claim 1, wherein said compound is selected from the group consisting of: a polynucleotide, a peptide, a peptidomimetic, a carbohydrate, a lipid, a small organic molecule and an inorganic molecule.

13. The method of claim 1, further comprising a step of applying a non-invasive brain stimulation (NIBS) to the subject.

14. The method claim 13, wherein said NIBS is selected from the group consisting of: electroconvulsive therapy (ECT), repetitive transcranial magnetic stimulation (rTMS), deep TMS, cranial electrotherapy stimulation (CES), transcranial direct current stimulation (tDCS), transcranial random noise stimulation (tRNS), and reduced impedance non-invasive cortical electrostimulation (RINCE).

15. A method for increasing the therapeutic response to NIBS therapy in a subject in need thereof, the method comprising administering to the subject a pharmaceutical composition comprising a therapeutically effective amount of at least one microglia modulator and at least one pharmaceutically acceptable carrier or diluent.

16. The method of claim 15, wherein an increased therapeutic response to NIBS is measured by a reduction in one or more effects selected from the group consisting of: acute confusional state, tachycardia, atrial arrhythmia, ventricular arrhythmia, hypertension, asystole, muscle pain, fatigue, headaches, nausea, and amnesia.

17. The method of claim 15, wherein an increased therapeutic response to NIBS is measured by a reduction in the number, length or frequency of NIBS treatments necessary to achieve a desired therapeutic effect, stimulus intensity, stimulus dosage necessary to achieve a desired therapeutic effect, or any combination thereof, or any combination thereof.

18. (canceled)

19. The method of claim 15, wherein said composition is administered 1 to 72 hours prior to applying NIBS.

20. The method of claim 15, wherein the ratio of microglia modulator administration to NIBS application ranges from 10:1 to 1:10, and optionally said NIBS is selected from the group consisting of: ECT, rTMS, CES, tDCS, tRNS, and RINCE.

21. (canceled)

22. The method of claim 15, wherein said subject is afflicted with a disorder selected from the group consisting of: unipolar major depressive episode, major depressive disorder, dysthymic disorder, treatment-resistant depression, bipolar depression, adjustment disorder with depressed mood, cyclothymic disorder, melancholic depression, psychotic depression, schizophrenia, post-schizophrenic depression, depression due to a general medical condition, post-viral fatigue syndrome, and chronic fatigue syndrome.

23. (canceled)

24. (canceled)

25. (canceled)

26. The method of claim 15, wherein said microglia modulator is an inhibitory-compound targeting a molecule selected from the group consisting of: LAG-3, CD180, TDO2, B7-2, PD-L1, and PLA2G4E.

27. The method of claim 1, wherein said microglia modulator is administered to the subject at a dosage of 0.01 to 100 mg/kg body weight.


SEQUENCE LISTING (18 sequences)

SEQ ID NO: 1 - DNA, 17 bp, Artificial Sequence
  Synthetic
tctccctcacaatttcc

SEQ ID NO: 2 - DNA, 17 bp, Artificial Sequence
  Synthetic
gggtgcagcgaacttta

SEQ ID NO: 3 - DNA, 22 bp, Artificial Sequence
  Synthetic
cttctggaagatggctcctaac

SEQ ID NO: 4 - DNA, 22 bp, Artificial Sequence
  Synthetic
ccctaagagagtggacaggata

SEQ ID NO: 5 - DNA, 20 bp, Artificial Sequence
  Synthetic
catcgtgtggtggtcatctt

SEQ ID NO: 6 - DNA, 22 bp, Artificial Sequence
  Synthetic
ctgatgctggagacaggtattc

SEQ ID NO: 7 - DNA, 22 bp, Artificial Sequence
  Synthetic
gacgttcagctactctgacttt

SEQ ID NO: 8 - DNA, 21 bp, Artificial Sequence
  Synthetic
gttggcctcttgtgttctttg

SEQ ID NO: 9 - DNA, 22 bp, Artificial Sequence
  Synthetic
tcatcacagtgactcccaaatc

SEQ ID NO: 10 - DNA, 22 bp, Artificial Sequence
  Synthetic
gccacacaaatctttcctttcc

SEQ ID NO: 11 - DNA, 22 bp, Artificial Sequence
  Synthetic
ctccgaaacctgtctcacttac

SEQ ID NO: 12 - DNA, 21 bp, Artificial Sequence
  Synthetic
gttctagctgagggcattctt

SEQ ID NO: 13 - DNA, 22 bp, Artificial Sequence
  Synthetic
ctccatcactcggctttcttat

SEQ ID NO: 14 - DNA, 22 bp, Artificial Sequence
  Synthetic
ctctcttccaagtgtccacaaa

SEQ ID NO: 15 - DNA, 21 bp, Artificial Sequence
  Synthetic
caggaacccatactgtgaaga

SEQ ID NO: 16 - DNA, 23 bp, Artificial Sequence
  Synthetic
gctggtaggagagtgtgataaat

SEQ ID NO: 17 - DNA, 21 bp, Artificial Sequence
  Synthetic
ccttaacactagaggcagcaa

SEQ ID NO: 18 - DNA, 20 bp, Artificial Sequence
  Synthetic
cattcaagcagcaggcattt